SEQUENCE LISTING (30 sequences)

SEQ ID NO: 1 - DNA, 18 bp, Artificial Sequence
  RNA-binding adaptor
agatcggaagagcacacg

SEQ ID NO: 2 - DNA, 28 bp, Artificial Sequence
  RNA-binding adaptor
  misc_feature (2)..(10)
  misc_feature (2)..(10): n is a, c, g, or t
annnnnnnnnagatcggaagagcacacg

SEQ ID NO: 3 - DNA, 30 bp, Artificial Sequence
  RNA-binding adaptor
  misc_feature (2)..(12)
  misc_feature (2)..(12): n is a, c, g, or t
annnnnnnnnnnagatcggaagagcacacg

SEQ ID NO: 4 - DNA, 28 bp, Artificial Sequence
  RNA-binding adaptor
  misc_feature (1)..(10): n is a, c, g, or t
  misc_feature (2)..(10)
nnnnnnnnnnagatcggaagagcacacg

SEQ ID NO: 5 - DNA, 28 bp, Artificial Sequence
  RNA-binding adaptor
  misc_feature (2)..(10)
  misc_feature (2)..(10): n is a, c, g, or t
annnnnnnnnagatcggaagagcacacg

SEQ ID NO: 6 - DNA, 18 bp, Artificial Sequence
  RNA-binding adaptor
agatcggaagagcacacg

SEQ ID NO: 7 - DNA, 30 bp, Artificial Sequence; identical to SEQ ID NO: 3
  RNA-binding adaptor
  misc_feature (2)..(12)
  misc_feature (2)..(12): n is a, c, g, or t

SEQ ID NO: 8 - DNA, 28 bp, Artificial Sequence
  RNA-binding adaptor
  misc_feature (1)..(10)
  misc_feature (1)..(10): n is a, c, g, or t
nnnnnnnnnnagatcggaagagcacacg

SEQ ID NO: 9 - DNA, 15 bp, Artificial Sequence
  RT primer
cgtgtgctcttccga

SEQ ID NO: 10 - DNA, 12 bp, Artificial Sequence
  RT primer
cgtgtgctcttc

SEQ ID NO: 11 - DNA, 27 bp, Artificial Sequence
  cDNA-binding adaptor
  misc_feature (2)..(8)
  misc_feature (2)..(8): n is a, c, g, or t
annnnnnnagatcggaagagcgtcgtg

SEQ ID NO: 12 - DNA, 26 bp, Artificial Sequence
  cDNA-binding adaptor
  misc_feature (1)..(7)
  misc_feature (1)..(7): n is a, c, g, or t
nnnnnnnagatcggaagagcgtcgtg

SEQ ID NO: 13 - DNA, 19 bp, Artificial Sequence
  cDNA-binding adaptor
agatcggaagagcgtcgtg

SEQ ID NO: 14 - DNA, 70 bp, Artificial Sequence
  Forward primer
aatgatacggcgaccaccgagatctacactatagcctacactctttccctacacgacgctcttccgatct

SEQ ID NO: 15 - DNA, 70 bp, Artificial Sequence
  Forward primer
aatgatacggcgaccaccgagatctacacatagaggcacactctttccctacacgacgctcttccgatct

SEQ ID NO: 16 - DNA, 70 bp, Artificial Sequence
  Forward primer
aatgatacggcgaccaccgagatctacaccctatcctacactctttccctacacgacgctcttccgatct

SEQ ID NO: 17 - DNA, 70 bp, Artificial Sequence
  Forward primer
aatgatacggcgaccaccgagatctacacggctctgaacactctttccctacacgacgctcttccgatct

SEQ ID NO: 18 - DNA, 70 bp, Artificial Sequence
  Forward primer
aatgatacggcgaccaccgagatctacacaggcgaagacactctttccctacacgacgctcttccgatct

SEQ ID NO: 19 - DNA, 70 bp, Artificial Sequence
  Forward primer
aatgatacggcgaccaccgagatctacactaatcttaacactctttccctacacgacgctcttccgatct

SEQ ID NO: 20 - DNA, 66 bp, Artificial Sequence
  Reverse primer
caagcagaagacggcatacgagatcgagtaatgtgactggagttcagacgtgtgctcttccgatct

SEQ ID NO: 21 - DNA, 66 bp, Artificial Sequence
  Reverse primer
caagcagaagacggcatacgagattctccggagtgactggagttcagacgtgtgctcttccgatct

SEQ ID NO: 22 - DNA, 66 bp, Artificial Sequence
  Reverse primer
caagcagaagacggcatacgagataatgagcggtgactggagttcagacgtgtgctcttccgatct

SEQ ID NO: 23 - DNA, 66 bp, Artificial Sequence
  Reverse primer
caagcagaagacggcatacgagatggaatctcgtgactggagttcagacgtgtgctcttccgatct

SEQ ID NO: 24 - DNA, 66 bp, Artificial Sequence
  Reverse primer
caagcagaagacggcatacgagatttctgaatgtgactggagttcagacgtgtgctcttccgatct

SEQ ID NO: 25 - DNA, 66 bp, Artificial Sequence
  Reverse primer
caagcagaagacggcatacgagatacgaattcgtgactggagttcagacgtgtgctcttccgatct

SEQ ID NO: 26 - DNA, 32 bp, Artificial Sequence
  Indexed P5 primer
aatgcacacactacacgacgctcttccgatct

SEQ ID NO: 27 - DNA, 27 bp, Artificial Sequence
  L5 adapter
  misc_feature (20)..(26)
  misc_feature (20)..(26): n is a, c, g, or t
gtgctgcgagaaggctagannnnnnna

SEQ ID NO: 28 - DNA, 28 bp, Artificial Sequence
  L7-cy5 adapter
  misc_feature (2)..(10)
  misc_feature (2)..(10): n is a, c, g, or t
annnnnnnnnagatcggaagagcacacg

SEQ ID NO: 29 - DNA, 12 bp, Artificial Sequence
  Universal reverse transcription primer
cttctcgtgtgc

SEQ ID NO: 30 - DNA, 32 bp, Artificial Sequence
  Indexed P7 primer
tctagccttctcgtgtgccagagtgtaggaac

(19) United States
(12) Patent Application Publication
(10) Pub. No.: US 2023/0227807 A1
(43) Pub. Date: Jul. 20, 2023

(54) METHOD FOR IDENTIFYING RNA BINDING PROTEIN BINDING SITES ON RNA

(71) Applicant: IMPERIAL COLLEGE INNOVATIONS LIMITED, London (GB)

(72) Inventor: Christopher Sibley, London (GB)

(21) Appl. No.: 17/997,787

(22) PCT Filed: May 7, 2021
(86) PCT No.: PCT/GB2021/051109

§ 371 Date: Nov. 2, 2022

(30) Foreign Application Priority Data
May 7, 2020 (GB) 2006803.7

(51) Int. Cl. C12N 15/10; C12Q 1/6806; C12Q 1/6876; C12Q 1/6869

(52) U.S. Cl. CPC C12N 15/1006; C12N 15/1096; C12Q 1/6806; C12Q 1/6876; C12Q 1/6869; C12Q 2600/16

(57) ABSTRACT

The invention relates to methods for purifying and isolating at least one RNA molecule which interacts with an RNA-binding protein (RBP). The invention also provides nucleic acid adaptors and primers for use in such methods.

### FIELD OF THE INVENTION

The invention relates to methods for purifying and isolating at least one RNA molecule which interacts with an RNA-binding protein (RBP). The invention also provides nucleic acid adaptors and primers for use in such methods.

### BACKGROUND OF THE INVENTION

RNA binding proteins (RBPs) are proteins that interact with RNA at specific sites, known as RNA-binding domains. RBPs play an essential role across cell physiology, as they are involved in regulating the fate of RNA molecules. This diverse group of proteins has been implicated in the modulation of pre-mRNA splicing, RNA modification, translation, stability and localisation.

A number of severe diseases are associated with, or can be caused by, disruption of the interaction between RBPs and RNA (e.g. Amyotrophic Lateral Sclerosis, myotonic dystrophy, various cancers). Targeting the interaction between RBPs and RNA, and thus modulating gene expression has potential therapeutic utility for the treatment and prevention of such diseases.

In silico approaches to identifying or predicting RBP-RNA interactions has proven challenging due to the various mechanisms in which RBPs may interact with RNA. A number of experimental strategies are, however, available for the identification and determination of RBP-RNA interactions in situ (i.e. in cell culture or animal models). The most widely used strategies for detecting direct RNA-protein interactions is the cross-linking and immunoprecipitation (CLIP) approach.

In principle, all CLIP workflows initiate with ultra-violet (UV) irradiation of the sample to induce covalent crosslinks between RBPs and their interacting RNA targets. This can be done either in vitro or in vivo such that crosslinking can capture a snapshot of the interactions at the time of cross-linking in most samples. Subsequently, an RBP-of-interest is purified before a nucleic acid adaptor is ligated to the partially digested RNA cargo in order to allow a sequencing compatible cDNA library to be produced. Crucially, the wavelength-specific selectivity of UV induced protein-RNA crosslinking makes it distinct from chemical crosslinking approaches that can also co-purify protein-DNA and protein-protein interactions. At least 28 distinct CLIP based protocols have now been reported. These primarily differ in the way in which they purify and visualise the RBP-RNA complex, or in the way they define positioning of the crosslinked nucleotide.

Of the 28 CLIP-based protocols, 24 of these involve visualisation of the purified RBP-RNA complexes before cDNA library construction, whilst 18 of the CLIP-based methods exploit reverse transcriptase stalling at the cross-linked nucleotide to identify the interaction site with single-nucleotide resolution. Visualisation of complexes represents an essential first quality control (QC) step of CLIP that can be used to i) assess presence and integrity of the purified complex against positive and negative control samples, ii) identify contaminating co-purified complexes (e.g. multi-mers, other RBPs), and iii) evaluate the RNase digestion conditions that impact integrity of downstream computational analysis. Meanwhile, although crosslink sites can be determined from limited cDNA read-through events with non-trivial computational methods, stalling of reverse transcription at the cross-linked nucleotide has been demonstrated both experimentally and computationally to occur at ^{˜}80-100% of all UV-induced crosslinking sites. Accordingly, CLIP methods that capture these truncations have potential to produce transcriptome-wide maps of protein-RNA interactions at single-nucleotide resolution that are capable of quantitative study.

CLIP complex visualisation has traditionally been carried out following an isotopic SDS-PAGE analysis, although the increasingly popular infrared-CLIP (irCLIP) approach introduced a non-isotopically labelled adaptor as a new means to visualise. Whilst this represents an attractive and safer alternative for most CLIP variants in principal, complexes identified by the published irCLIP protocol display distinct and intense bands that appear at common sizes despite diverse molecular weights of the profiled proteins. Interestingly, these bands remain in negative controls absent from initial publication, thus making assessment of key experimental variables non-trivial. Such observations are inconsistent with previous well-used isotopic methods, and suggest that the integrity of the irCLIP method has scope for improvement. Meanwhile, whilst most protocols still include complex visualisation, the most notable CLIP protocol that excludes complex visualisation is the enhanced CLIP (eCLIP) protocol used by ENCODE, among others. Here speed and scalability is prioritised over the complication of radiolabelling hundreds of RBPs. Indeed, whilst integrity of few eCLIP immunoprecipitations have been validated with isotopic labelling, predicted complexes are isolated blindly based on their western blot assessed molecular weight. This is despite the fact that many antibodies work inefficiently for CLIP despite working well in westerns, whilst targeting a single RBP under standard conditions can sometimes co-precipitate other RBPs or isolate macromolecular complexes.

Aside from these considerations, irCLIP and eCLIP represent expedited variants of the individual nucleotide resolution CLIP (iCLIP) approach that first exploited cDNA truncations to identify sites of crosslinking. Indeed, whilst iCLIP consistently produces high quality cDNA libraries alongside comprehensive quality controls, the protocol is lengthy, being carried out over 6 days. Furthermore, the iCLIP methodology is technically challenging. The time required and the technical challenges limit the take up and utility of iCLIP. Both irCLIP and eCLIP introduced new adaptations to the iCLIP protocol that lead to reproducible improvements in efficiency at certain steps, such that both protocols take around 3-4 days. However, recent computational comparisons suggest that iCLIP still remains the gold standard in terms of data quality, determination of RBP-occupancy, and quantitative capabilities. However, even with iCLIP there are technical challenges and issues regarding the integrity of the resulting data, even leaving aside the undesirable time required to complete the protocol.

With the above in mind, and in view of the limitations and disadvantages of the currently available CLIP-based protocols, there is a significant need for an improved CLIP-based methodology for the robust, efficient and high-resolution analysis of RBP-RNA interactions and to accurately determine RBP-binding sites on RNA transcripts. This also has the potential to accurately identify new drug targets for diseases where perturbations in RBP-RNA interactions are a contributing factor.

### SUMMARY OF THE INVENTION

The present invention provides enhanced CLIP-based methods and products for use in such methods.

The present inventors have developed a robust, simple and non-isotopic enhanced iCLIP (eiCLIP) protocol that produces highest quality cDNA libraries in as little as two days. The method developed by the inventors allows the complete removal of experimental artefacts often associated with conventional CLIP protocols without cumbersome and inefficient gel-based size selection. Importantly, the protocol retains key QC steps to assess and optimise experimental integrity, whilst its efficiency permits a smaller test sample (as few as 10,000 cells) to be used as starting input. The present inventors have also developed novel nucleic acid adaptors for use herein in their new eiCLIP-based methods which prevent non-specific binding within the mixtures, resulting in improved visualisation of cross-linked RBP-RNA, free of experimental artefact. In addition, adaptors of the present invention are significantly more cost-effective to synthesise and have improved yield over the conventional adaptors used in CLIP-based methods.

Advantageously, methods of the invention can be used to produce sequencing ready cDNA libraries in as little as two days. In addition, the quantity of starting material necessary has been greatly reduced by employing the efficient and streamlined methods of the invention.

Accordingly the invention provides a method for purifying at least one RNA molecule which interacts with one or more target RNA binding protein, (RBP) comprising the steps of: (a) cross-linking the at least one RNA molecule and the one or more RBP in a sample; (b) contacting the sample comprising the cross-linked RBP-RNA with an agent which cleaves RNA to create a first mixture, wherein said agent shortens the RPB-bound RNA; (c) purifying the cross-linked RBP-RNA from the first mixture using an agent that specifically interacts with a component of the cross-linked RBP-RNA; (d) contacting the purified cross-linked RBP-RNA from step c with an RNA-binding adaptor comprising a detection means to create a second mixture, wherein the adaptor binds to the cross-linked RNA; (e) removing any unbound RNA-binding adaptor by contacting the second mixture with a 5′ to 3′ exonuclease (e.g. RecJ); (f) isolating the adaptor-bound cross-linked RBP-RNA; and (g) visualising the cross-linked RBP-RNA by detection of the detection means; thereby purifying at least one RNA molecule which interacts with the one or more target RBP.

Said method of claim may further comprise the steps of: (h) partially digesting the RBP component of the cross-linked RBP-RNA, optionally using a proteinase; (i) purifying the at least one RNA molecule; and (j) preparing the at least one RNA molecule for high throughput sequencing.

The agent which specifically interacts with a component of the cross-linked RBP-RNA in step c may be: (i) an antibody which specifically binds to an RBP of interest; (ii) an antibody which specifically binds to a modification of the RNA of interest; or (iii) a nucleic acid molecule that is homologous to an RNA sequence of interest.

A portion of the first mixture may be removed immediately after step b and the whole proteome from said portion captured using an agent that specifically interacts with protein side chains to provide an input control. The portion of the first mixture removed may be about 10%, about 5% or about 1% of the total volume of said first mixture, preferably about 5%; and/or the input control may be processed in parallel to the remainder of the first mixture.

The invention also provides a method for isolating a plurality of RNA molecules interacting with all RBP contained in a sample, comprising the steps of: (a) cross-linking the plurality of RNA molecules and the RBP in the sample; (b) contacting the sample comprising the cross-linked RBP-RNA with an agent which cleaves RNA to create a first mixture, wherein said agent shortens the RPB-bound RNA; (c) purifying the cross-linked RBP-RNA from the first mixture using an agent that specifically interacts with protein side chains; (d) contacting the purified cross-linked RBP-RNA from step c with an RNA-binding adaptor comprising a detection means to create a second mixture, wherein the adaptor binds to the cross-linked plurality of RNA molecules; (e) removing any unbound adaptor by contacting the second mixture with a 5′ to 3′ exonuclease; (f) isolating the adaptor-bound cross-linked RBP-RNA; and (g) purifying the plurality of RNA molecules; wherein optionally said method further comprises: a step of visualising the cross-linked RBP-RNA by detection means between steps (f) and (g) and/or the steps of: (h) partially digesting the RBP component of the cross-linked RBP-RNA, optionally using a proteinase; (i) purifying the at least one RNA molecule; and (j) preparing the at least one RNA molecule for high throughput sequencing.

The agent which specifically interacts with protein side chains may comprise a carboxyl group.

The sample may be a sample comprising cells. Optionally, in such methods, a further step of lysing the cells to produce a cell lysate, wherein said lysis is performed immediately before step (b).

The cross-linking may be UV cross-linking.

The agent which cleaves RNA may be a ribonuclease, preferably RNase I.

The agent which specifically interacts with a component of the cross-linked RBP-RNA or the agent that specifically interacts with protein side chains in step c may be immobilised on a solid phase, and wherein optionally said solid phase comprises magnetic beads.

Any method of the invention may further comprise a washing step under stringent conditions: (i) immediately after step c; (ii) immediately after step d; and/or (iii) immediately after step e.

The RNA-binding adaptor may be between 18 and 32 nucleotides in length.

The detection means may be a fluorophore/fluorescent detection means, preferably a cyanine, more preferably a cyanine with an excitation wavelength of about 675 nm and an emission wavelength of about 694 nm.

The RNA-binding adaptor may comprise or consist of a nucleotide sequence selected from:

(SEQ ID NO: 1)
AGATCGGAAGAGCACACG;
(SEQ ID NO: 2)
A[XXXXXX]NNNAGATCGGAAGAGCACACG;
(SEQ ID NO: 3)
A[XXXXXXXX]NNNAGATCGGAAGAGCACACG;
(SEQ ID NO: 4)
N[XXXXXX]NNNAGATCGGAAGAGCACACG;
(SEQ ID NO: 5)
AGATCGGAAGAGCACACG/3Cy55Sp/;
(SEQ ID NO: 6)
A[XXXXXX]NNNAGATCGGAAGAGCACACG/3Cy55Sp/;
(SEQ ID NO: 7)
A[XXXXXXXX]NNNAGATCGGAAGAGCACACG/3Cy55Sp/;
(SEQ ID NO: 8)
N[XXXXXX]NNNAGATCGGAAGAGCACACG/3Cy55Sp/.

The RNA-binding adaptor may be 5′ adenylated, and optionally a deadenylase is used in combination with a 5′ to 3′ exonuclease to remove any unbound RNA-binding adaptor.

The 5′ to 3′ exonuclease may be RecJ, preferably RecJ_{f}.

The method for purifying at least one RNA molecule which interacts with one or more target RNA binding protein, or the method for isolating a plurality of RNA molecules interacting with all RBP contained in a sample, may comprise a step of preparing the RNA molecules for high throughput sequencing which optionally comprises: (i) reverse transcription of the RNA molecules to produce a plurality of cDNA molecules; (ii) enzymatic digestion of any unextended reverse transcription primer; (iii) immobilisation of the plurality of cDNA molecules on a solid phase; (iv) ligation of a cDNA-binding adaptor to the immobilised plurality of cDNA molecules; (v) optionally eluting the plurality of cDNA molecules from the solid phase; and (vi) amplification of the plurality of cDNA molecules; wherein optionally the step of preparing the RNA molecules for high throughput sequencing further comprises a step of alkaline hydrolysis to remove the RNA molecules, wherein the step of alkaline hydrolysis is performed between (i) and (ii).

The invention also provides a method of preparing one or more RNA molecule for high-throughput sequencing comprising: (i) reverse transcription of the one or more RNA molecule to produce a plurality of cDNA molecules; (ii) enzymatic digestion of any unextended reverse transcription primer; (iii) immobilisation of the plurality of cDNA molecules on a solid phase; (iv) ligation of a cDNA-binding adaptor to the immobilised plurality of cDNA molecules; (v) optionally eluting the plurality of cDNA molecules from the solid phase; and (vi) amplification of the plurality of cDNA molecules; wherein optionally the one or more RNA molecule is prepared by the method of any one of claims 1 to 18.

The reverse transcription may use a revere transcription primer that is a universal biotinylated reverse transcription primer, wherein optionally: (i) said primer comprises a nucleic acid sequence selected from CGTGTGCTCTTCCGA (SEQ ID NO: 9) or CGTGTGCTCTTC (SEQ ID NO:10) (ii) said primer is biotinylated at the 5′ end; and/or (iii) the oligonucleotide sequence of said primer is separated from the biotin moiety by a linker, preferably tetraethyleneglycol (TEG).

The enzymatic digestion of any unextended reverse transcription primers may be carried out using Exonuclease III digestion.

The plurality of cDNA molecules may be immobilised using magnetic streptavidin beads.

The plurality of cDNA molecules may be eluted from the solid phase in nuclease-free and metal ion-free water at a temperature of at least 50° C.

The amplification of the plurality of cDNA molecules may be carried out by PCR using indexed reverse primers modified with 3 phosphorothioate bonds at the 3′ end.

Preparing one or more RNA molecule for high-throughput sequencing may further comprise purification of the amplified plurality of cDNA molecules. Preparing one or more RNA molecule for high-throughput sequencing may further comprise exonuclease III digestion of any unextended reverse transcription primers and PCR amplification of the plurality of cDNA molecules using indexed reverse primers modified with 3 phosphorothioate bonds at the 3′ end.

Any method of the invention may further comprise carrying out high throughput sequencing on the purified cDNA.

The invention also provides an RNA-binding adaptor comprising a detection means, as defined herein.

The invention also provides a universal biotinylated reverse transcription primer as defined herein.

The invention further provides a kit comprising: (i) an RNA-binding adaptor as defined herein; and/or (ii) a universal biotinylated reverse transcription primer as defined herein; and instructions for using said RNA-binding adaptor and/or primer in a method of cross-linking immunoprecipitation (CLIP)

The invention further provides the use of an RNA-binding adaptor of the invention and/or a universal biotinylated reverse transcription primer of the invention in a method of cross-linking immunoprecipitation (CLIP).

The invention also provides a method for screening molecules which disrupt the interaction of at least one RNA molecule with one or more target RBP, comprising the steps of: (i) treating a sample with a molecule which disrupts protein-RNA interactions; (ii) carrying out the method of the invention on the treated sample; and (iii) comparing the treated sample with an untreated control sample. Optionally said method is used to screen molecules for treating a disease or disorder associated with one or more target RBP.

### BRIEF DESCRIPTION OF THE DRAWINGS

FIG. 1: Flow chart of an exemplary enhanced iCLIP (eiCLIP) protocol of the invention for the detection of RBP-RNA interactions. A) Summarised protocol demonstrating experimental steps. B) Oligonucleotide adapter/primer designs used in eiCLIP.

FIG. 2: Optimised CLIP parameters for non-isotopic RBP-RNA complex detection. A) irCLIP of PTBP1 demonstrating unexpected banding that masks the signal indicating detection of PTBP1 complexes with RNA. B) Final PCR library from irCLIP experiments leads to an adapter specific artefact that dominates libraries and is present in negative control samples. C) SFPQ irCLIP reveals double banding around the molecular weight of the protein. The lower band is additionally observed in the no UV condition to indicate adapter attachment to immunoprecipitated protein without RNA ligation. D) Increased washing and in-lysate digestions improves irCLIP detection of PTBP1 complexes with RNA. E) Designs of irCLIP and eiCLIP non-isotopic RNA adapters. F) Optimised eiCLIP conditions reveal complexes of multiple RBPs with their interacting RNAs with high integrity and absence of unintended banding caused by adapter attachment in absence of RNA ligation. Left two panels (PTBP1, NONO) include optimised irCLIP conditions for direct comparison. * on right panel (SFPQ) indicates signal derived from co-immunoprecipitated RBP. G) To mitigate gel-based size selection in eiCLIP, optimisation of RNA fragment length in eiCLIP is achieved by initial optimisation of RNase I digestion conditions on sample lysates. In this panel the RNA was extracted from the size matched input of samples treated with differing amounts of RNase I and analyzed by gel electrophoresis and membrane transfer.

FIG. 3: Removal of adapter-specific artefacts in eiCLIP library preparation steps. A) Free adapter entering the library preparation can be processed into a library artefact that has potential to dominate libraries (1). This can be partially removed by exonuclease III digestion of the free adapter annealed to its reverse complement (2), and use of phosphorothioate modified primers in final PCRs (3). Combination of these two steps (4), together with RecJ_{f} removal of free adapter following initial ligation, is able to remove potential PCR artefact from libraries. B) eiCLIP final PCR libraries are absent of artefact bands in negative control samples, and evident of desired diverse cDNA lengths in replicate samples.

FIG. 4: An improved Size Matched Input (SMI) in the eiCLIP protocol. A) Non-isotopic imaging of 5% input lysates from HeLa cells incubated with SP3 paramagnetic beads and processed through the eiCLIP protocol under optimal RNase conditions reveals protein-RNA complex signal across a diverse range of molecular weights. This is indicative of multiple RNA-binding proteins being captured and contributing signal in each sample. Immuno-labelling of 5% input lysates incubated with SP3 paramagnetic beads confirms the retention of RNA-binding proteins of diverse sizes. This is in addition to non-RNA binding proteins present within the cells proteome. B) Cross-linking profiles of the SMI are distinct from the eiCLIP of specific RNA-bindings proteins such as hnRNP C in HeLa cells. Shown is the CD55 locus, with boxed regions highlighting regions of distinct crosslinking between hnRNP C eiCLIP replicates and the corresponding SMI eiCLIP replicates taken from identical lysates.

FIG. 5: Comparison of eiCLIP to other related methods. A) Comparison of eiCLIP, iCLIP, irCLIP and eCLIP crosslinking at the CD55 locus validated in previous iCLIP studies. B) Percentage crosslinking of different hnRNP C libraries at different transcriptome features. C) Correlations of transcriptome-wide crosslinking at high confidence hnRNP C iCLIP clusters (>15 iCLIP reads per cluster) in eiCLIP and irCLIP methods. Note, eCLIP is not included in this analysis due to a different background cell line being used to generate the publicly available hnRNP C eCLIP datasets. D) Comparison of eiCLIP crosslinking at the at the CD55 locus using different amounts of cells as starting input. U2AF65 derived eiCLIP crosslinking sites and hnRNP C derived iCLIP crosslinking sites are included for comparison. E) Comparison of eiCLIP crosslinking at a validated hnRNP C binding site within the CD55 locus using different amounts of cells as starting input. U2AF65 derived eiCLIP crosslinking sites and hnRNP C derived iCLIP crosslinking sites are included for comparison.

### DETAILED DESCRIPTION OF THE INVENTION

Unless otherwise defined herein, scientific and technical terms used in connection with the present invention shall have the meanings that are commonly understood by those of ordinary skill in the art. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

The publications discussed herein are provided solely for their disclosure prior to the filing date of the present application. Nothing herein is to be construed as an admission that such publications constitute prior art to the claims appended hereto.

Methods of Purifying and/or Isolating RNA

The methods of the present invention comprise a step of cross-linking at least one RNA molecule with one or more RBPs. Cross-linking forms one or more bonds (e.g. covalent or ionic) which links the at least one RNA molecule with one or more RBPs. In the context of the present invention, typically said bonds are covalent bonds. Cross-linking of the at least one RNA molecule with the one or more RBP allow rigorous methods to be employed to purify the RBP-RNA complex from a sample. Advantageously, cross-linking of the at least one RNA molecule with the one or more RBP enables the partial cleavage and shortening of RNA molecules using nucleases, without disrupting the RBP-RNA interactions. Typically the cross-linking is induced by irradiating the sample with ultra-violet (UV) radiation. Alternatively, a chemical cross-linker, preferably methylene blue (methylthioninium chloride), may be used to cross-link at least one RNA molecule with one or more RBPs. By way of example, methylene blue may be added to a sample comprising RNA and RBPs and the sample irradiated with visible light (i.e. light with a wavelength of between 380 to 800 nm). Preferably, the cross-linking is induced by irradiating the sample with UV radiation at a wavelength of about 254 nm. Cross-linking at 365 nm following 4SU exposure is also encompassed. UV radiation induces the formation of covalent cross-links between RBPs and RNA only at sites of direct contact between RBPs and RNA. Cross-linking of only those direct interactions between RBPs and RNA allows single-nucleotide resolution identification of the RBP interaction site.

The precise UV parameters necessary to induce cross-linking between RBPs and RNA are well known to the skilled person. The skilled person will also understand the precise UV parameters may need to be adjusted depending on the type of sample being irradiated (for example, cells or tissue). Typically, the amount of UV energy used to induce cross-linking will be between 25 to 500 mJ/cm², preferably between 100 to 400 mJ/cm². By way of non-limiting example, cross-linking may be induced by irradiating a sample with 150 mJ/cm². Tissue samples undergoing UV cross-linking may require multiple exposures, for example, three exposures of 100 mJ/cm². The UV exposure time will typically depend on the energy used, and can be readily determined by the skilled person. By way of example, using 150 mJ/cm², an exposure time of about 45 seconds may be used.

Methods of the invention may comprise a step of introducing a photoreactive nucleoside into living cells, wherein the living cells incorporate the photoreactive nucleoside into an RNA molecule during transcription. As used herein, the term “photoreactive nucleoside” refers to a modified nucleoside that contains a photochromophore and is capable of cross-linking with an RBP. By way of non-limiting example, the photoreactive nucleoside may be a thiouridine analogue, such as 2-thiouridine, 4-thiouridine or 2,4-dithiouridine, or a thioguanisine analogue, such as 6-thioguanisine. The step of introducing a photoreactive nucleoside into living cells may be performed before the step of cross-linking the at least one RNA molecule and the one or more RBP in a sample. In embodiments involving photoreactive nucleosides, cross-linking of the at least one RNA molecule with the one or more RBP is induced by irradiating the sample with UV radiation. Preferably, the cross-linking is induced by irradiating the sample with UV radiation at a wavelength of 365 nm.

The methods of the present invention comprise a step of contacting the sample comprising the cross-linked RBP-RNA with an agent which cleaves RNA to create a first mixture, wherein said agent shortens the RPB-bound RNA. The term “shortening the RPB-bound RNA” is interchangeable with the term “partial digestion of the RPB-bound RNA”, and involves cleavage of the RNA molecule to remove one or more nucleic acid residue. Cleavage of the RNA molecule following cross-linking generate RBP-bound RNA fragments that are suitable for downstream analysis. For example, sequencing, particularly high throughput short-read sequencing, is compatible with shorter fragments. Shortening the RNA also cuts the RNA so that RBP further along the transcripts are not co-purified. The expression “shortens the RBP-bound RNA” is intended to encompass the removal of at least one nucleotide from the RBP-bound RNA. As the skilled person will appreciate, the removal of at least one nucleotide from the RBP-bound RNA will occur in regions of the RNA molecule not cross-linked to an RBP. The shortening may remove at least one nucleotide from the RBP-bound RNA, preferably at least two, at least three, at least four, at least five, at least six, at least seven, at least eight, at least nine, at least 10, at least 20, at least 30, at least 40, at least 50, at least 60, at least 70, at least 80, at least 90 or at least 100 nucleotides from the RBP-bound RNA. The shortening may occur at the 3′ end of the RNA molecule, the 5′ end of the RNA molecules, or both the 3′ and the 5′ ends of the RNA molecule. The shortening/partial digestion step may remove all of the RNA molecules that are not cross-linked to an RBP. A method of the invention may comprise a step of contacting the sample comprising the cross-linked RBP-RNA with at least one nuclease capable of cleaving the RNA molecule into fragments and shortening the RPB-bound RNA. Preferably, the at least one nuclease is an endoribonuclease, for example ribonuclease I (RNase I) which may be isolated from Escherichia coli. RNase I preferentially hydrolyses single-stranded RNA to nucleoside 3′-monophosphatse via nucleoside 2′, 3′-cyclic monophosphate intermediates. This leads to a 5′ hydroxyl group and a 3′ phosphate group. The 5′ hydroxyl group acts as a block to prevent self-circularisation of the RNA molecule(s) when ligating the adaptor. The 3′ phosphate may be modified to a 3′ hydroxyl by means of a de-phosphorylation reaction prior to ligation of the adaptor. Typically the step of shortening the RNA results in each RBP-bound RNA being cleaved to between 19 to 1000 nucleotides in length to facilitate downstream processing, such as high throughput sequencing.

The methods of the present invention comprise a step of purifying the cross-linked RBP-RNA from the first mixture using an agent that specifically interacts with a component of the cross-linked RBP-RNA. The terms “purifying” and “isolating” as used interchangeably herein. As used herein, the term “purifying” refers to a process well known to those of skill in the art in which components of a complex mixture are substantially separated from other components in the mixture. As a non-limiting example, purification of the cross-linked RBP-RNA may remove at least 80%, at least 85%, at least 90%, at least 95%, at least 96%, at least 97%, at least 98%, at least 99% or more, up to 100% of the other components (e.g. proteins, DNA, non-RBP-bound RNA, cell membrane fragments and/or other cellular debris) of the first mixture.

According to the present invention, cross-linked RBP-RNA is purified from the first mixture using an agent that specifically interacts with a component of the cross-linked RBP-RNA. For example, the cross-linked RBP-RNA may be purified using an agent that specifically interacts with the RNA molecule or the RBP.

The agent may specifically interact with the RBP component of the RBP-RNA complex. Agents that specifically interact with RBPs are well known to the skilled person. By way of non-limiting example, antibodies and antigen-binding fragment thereof or aptamers are both capable of specific interaction with RBPs and may be used to purify the cross-linked RBP-RNA from the first mixture. The use of antibodies or other agents which specifically interact with an RBP of interest are particularly useful when a method of the invention is used to purify/isolate RNA which binds to one or more particular RBPs of interest. Other non-limiting examples include the use of an RBP comprising a tag which can be used to assist in purification of the RBP-RNA complexes. Such tagged RBP may be used when a method of the invention is used to identify the RNA sequences which bind to a particular known RBP of interest. By way of a further non-limiting example, a complementary oligonucleotide to the adaptor which binds to the RBP-bound RNA could be used, particularly in instances where the adaptor is bound to a solid support (e.g. a magnetic bead).

Purifying the cross-linked RBP-RNA using an agent that specifically interacts with the RBP component allows more streamlined protocols for downstream RNA sequence analysis to be employed. Thus, the agent that specifically interacts with the RBP component of the cross-linked RBP-RNA may be an antibody or antigen-binding fragment thereof. Preferably, the agent that specifically interacts with the RBP component of the cross-linked RBP-RNA is an antibody or antigen-binding fragment thereof which specifically interacts with an RBP of interest or which specifically binds to a modification of the RNA of interest. Different antigen-binding fragments of preferred antibodies, e.g. Fab fragments, scFvs, diabodies or single domain antibodies are encompassed by the term “antibody or antigen-binding fragment thereof” as used herein, and can be readily obtained using conventional techniques.

Alternatively, the agent may specifically interact with proteins, but not other cellular components (e.g. DNA/RNA). Examples of such agents include agents which specifically interact with protein side chains, e.g. agents comprising one or more carboxyl groups. Such agents are typically used when a method of the invention is used to isolate the RNA molecules interacting with all the RBP in a sample. By way of non-limiting example, carboxylic acid coated magnetic beads provide a non-specific (affinity) capture of all RBPs within a sample. The use of magnetic beads provides an efficient means of isolating RBP/RNA complexes from a sample.

The agent may specifically interact with the RNA component of the RBP-RNA complex. Agents that specifically interact with RNA are well known to the skilled person. By way of non-limiting example, nucleic acid or peptide nucleic acid molecule may be designed to specifically interact with the RNA component of the cross-linked RBP-RNA. Universal nucleic acid agents may be used. Alternatively, nucleic acid agents specific for particular RNA molecules of interest may be used. Nucleic acid agents may be designed based on sequence homology with target RNA molecules. Purifying the cross-linked RBP-RNA using an agent that specifically interacts with the RNA component is particularly useful for determining which RBP(s) bind to particular RNA sequence or region. Thus, the agent that specifically interacts with the RNA component of the cross-linked RBP-RNA may be a nucleic acid. Preferably, the nucleic acid molecule is complimentary to an RNA sequence of interest.

The agent that specifically interacts with a component of the cross-linked RBP-RNA may be immobilised on a solid support, such as in the form of a column or beads. Said beads may be magnetic beads, deformable beads (e.g. agarose beads), or silica beads. Preferably said beads are magnetic. Capture agents, such as biotin/streptavidin, which can be captured using a second agent (e.g. streptavidin where the first capture agent is biotin, or biotin where the first capture agent is streptavidin) or antibodies (or antigen-binding fragments thereof) may be chemically linked to a solid support. Divalent metal ions (for example, Ni, Co ad Cu) may also be used as capture agents. Typically, divalent metal ions are chelated to a solid support, such as a silica resin or agarose bead, and used in the affinity capture of proteins (e.g. RBPs). This process may be referred to as immobilized metal affinity chromatography (IMAC). By way of non-limiting example, Ni may be used in the affinity purification of polyhistidine-tagged RBPs. This example would be particularly useful for determining which RNA molecules(s) bind to a particular RBP.

The methods of the present invention comprise a step of contacting the purified cross-linked RBP-RNA with an RNA-binding adaptor comprising a detection means to create a second mixture, wherein the RNA-binding adaptor binds to the cross-linked RNA. As used herein, the term “RNA-binding adaptor”, refers to an oligonucleotide that is capable of being ligated to the 3′ end of the RBP-bound RNA molecule. The RNA-binding adaptor may be DNA or RNA. Typically, the RNA-binding adaptor is a single-stranded oligonucleotide. Preferably, the RNA-binding adaptor is composed of DNA nucleotides. The term “detection means” is intended to encompass a detectable label attached to the RNA-binding adaptor during oligonucleotide synthesis which allows the detection of the cross-linked RBP-RNA once the RNA-binding adaptor has been ligated to the RNA component of the cross-linked RBP-RNA. The skilled person will be well aware of the various detection means used in molecule biology. By way of non-limiting example, the detection means may be fluorescent detection means, radioactive detection means, chemiluminescent detection means, or immunological detection means (for example, digoxigenin (DIG) may be conjugated to the RNA-binding adaptor and detected with labelled anti-DIG antibodies). Preferably the detections means is a fluorescent detection means. One of skill in the art will understand that any fluorescent tag or label can be covalently attached to an oligonucleotide in order to aid the detection of the oligonucleotide. Near infra-red fluorophores are particularly useful in methods of the present invention, for example, fluorescent detection means having excitation wavelengths of about between 650 nm and 800 nm and emission wavelength of about between 660 nm to 850 nm. The fluorescent detection means may be a cyanine or an Alexa Fluor dye (e.g. Alexa Fluor 660, 680, 700, 750 or 790). A particularly preferred fluorescent detection means is a cyanine with an excitation wavelength of about 675 nm and an emission wavelength of about 694 nm. An exemplary fluorescent detection means according to the invention is Cy5.5, particularly Cy5.5 incorporated at the 3′ end of the RNA-binding adapter. Typically, the fluorescent detection means is not, or does not comprise, IRDye 800CW DBCO.

Standard adaptors used and visualised in conventional CLIP protocols are ‘sticky’, such that they attach to any component in the ligation reaction (e.g. enzymes, the RBP, antibodies), even if said component is not ligated to the RNA as intended. This manifests during the step of visualising the cross-linked RBP-RNA as striated bands in the SDS-PAGE analysis, resulting in a poor ability to visualise and QC the RBP-RNA complexes that are being isolated and profiled. Notably, these bands are at common sizes across experiments of RBPs with different molecular weights, whilst they also appear in negative controls. They are therefore not specific and require removal. Indeed, carry over into subsequent steps leads to a single dominant artefact resulting from processing of the unligated adapter which hinders experimental interpretation.

Advantageously, the present inventors have developed RNA-binding adaptors of up to 35 nucleotides in length that reduce aberrant binding to non-RNA component of the sample and provide improved visualisation compared to conventional adaptors used in CLIP-based protocols. The synthesis yield of RNA-binding adaptors according to the present invention is also higher and more cost-effective.

Typically the RNA-binding adaptor of the invention is at least 10 nucleotides in length. For example, the RNA-binding adaptor may be 10, 11, 12, 13, 14, 15, 16, 17, 18, 19, 20, 21, 22, 23, 24, 25, 26, 27, 28, 29, 30, 31 or 32 nucleotides in length. Preferably the RNA-binding adaptor is between 15 and 35 nucleotides in length, more preferably between 18 and 35 nucleotides in length, even more preferably between 18 and 32 nucleotides in length.

Thus, typically the RNA-binding adaptor has an adenine nucleotide at its 5′ position. The provision of RNA-binding adaptors, all with the same 5′ nucleotide, reduces ligation bias in any downstream sequencing steps. The RNA-binding adaptor may comprise a nucleotide sequence selected from: AGATCGGAAGAGCACACG (SEQ ID NO: 1); A[XXXXXX]NNNAGATCGGAAGAGCACACG (SEQ ID NO: 2); or

A[XXXXXXXX]NNNAGATCGGAAGAGCACACG (SEQ ID NO: 3). Alternatively, the RNA-binding adaptor may have any other nucleotide at its 5′ position. Such an adaptor may comprise the following nucleotide sequence: N[XXXXXX]NNNAGATCGGAAGAGCACACG (SEQ ID NO: 4). Each instance of X and N may be independently selected from any nucleic acid.

The RNA-binding adaptor may comprise an index section, a barcode section, or both an index section and a barcode section. Preferably the RNA-binding adaptor of the invention comprises an index region and a random barcode region. The index section may be defined as a nucleotide sequence of known base composition, but where this composition varies between different versions of the adapter. In other words, the index section may be defined as a stretch of nucleotides of known sequence. The sequence of the index section may vary between each adapter. The inclusion of an index section of known sequence within an RNA-adapter of the invention allows for sample mixing to occur post-ligation which reduces any technical variability seen. Thus, the index section may comprise from five to ten nucleic acid resides, and typically comprises from five to eight nucleic acid residues, preferably six, seven or eight nucleic acid residues. The barcode section may be defined as a unique molecular identifier composed of a specified length of nucleotides of random sequence composition. Thus, the barcode section may comprise from two to ten random nucleic acid resides, and typically comprises from two to five random nucleic acid residues, preferably three random nucleic acid residues. Thus, exemplary consensus sequences comprised by an RNA-binding adaptor of the invention are A[XXXXXX]NN NAGATCGGAAGAGCACACG (SEQ ID NO: 2), A[XXXXXXXX]NN NAGATCGGAAGAGCACACG (SEQ ID NO: 3), and N[XXXXXX]NNNAGATCGGAAGAGCACACG (SEQ ID NO: 4), where X is a nucleic acid residue of the index section and N is a nucleic acid of the barcode section. Each instance of X and N may be independently selected from any nucleic acid.

Thus, preferred RNA-binding adaptors of the invention include SEQ ID NOs: 1 to 4 with a 3′ Cy5.5 tag:

(SEQ ID NO: 5)
A[XXXXXX]NNNAGATCGGAAGAGCACACG/3Cy55Sp/;
(SEQ ID NO: 6)
AGATCGGAAGAGCACACG/3Cy55Sp/;
(SEQ ID NO: 7)
A[XXXXXXXX]NNNAGATCGGAAGAGCACACG/3Cy55Sp/;
and
(SEQ ID NO: 8)
N[XXXXXX]NNNAGATCGGAAGAGCACACG/3Cy55Sp/.

The skilled person will understand that where the preferred RNA-binding adaptors are composed of RNA, the thymine nucleotides will be replaced by uracil nucleotides.

The methods of the present invention may comprise a step of removing any unligated RNA-binding adaptor by contacting the second mixture with a 5′ to 3′ exonuclease. Removal of any unligated RNA-binding adaptor eliminates artefacts from the sample thus improving the integrity of subsequent visualisation steps compared with conventional methods, such as irCLIP. The use of an exonuclease therefore typically reduces the amount of residual “free” adaptor in cDNA libraries produced using the methods of the invention, resulting in libraries that only contain immunoprecipitated RNA, rather than adaptor-specific by-products. Exonucleases with 5′ to 3′ activity are well known to those skilled in the art and include, for example, RecJ, Exonuclease VIII, lambda exonuclease and T5 exonuclease. To ensure that only unligated RNA-binding adaptors are removed from the second mixture, typically the 5′ to 3′ exonuclease is single stranded DNA-specific. Ligated RNA-binding adaptors have their 5′ end bound to the 3′ end of the RNA molecule to which they have been ligated and are thus protected from the actions of such an exonuclease. In contrast, unbound RNA-binding adaptors have a phosphorylated 5′ end that serves as the substrate for the single stranded DNA specific exonuclease. Preferably, the exonuclease is RecJ. Thus, any of the disclosure herein which refers to a 5′ to 3′ exonuclease explicitly encompasses the use of RecJ. The term “RecJ” as used herein refers to the single stranded DNA-specific exonuclease encoded by the RecJ gene in Escherichia coli (NCBI Reference Sequence: NP_417368.1 (deposited 11 Oct. 2018), Gene ID: 947367, Genomic sequence: NC_000913.3). RecJ catalyses the removal of deoxy-nucleotide monophosphates from single stranded DNA in the in the 5′ to 3′ direction. Recif, which is a fusion of RecJ and maltose binding protein (which improves the solubility of RecJ) may preferably be used. Variants and fragments of RecJ which retain the exonuclease activity of wild-type RecJ may also be used. The optimal conditions for RecJ activity are well established. By way of non-limiting example, unligated adaptor may be removed by contacting the second sample with 15 units of RecJ, optionally in a buffer comprising 50 mM NaCl, 10 mM Tris-HCl, 10 mM MgCl₂ and 1 mM DTT for 30 minutes at 37° C.

The 5′ end of the RNA-binding adaptor may be adenylated, i.e. it may have a 5′-adenylpyrophosphoryl cap. The RNA-binding adaptor may be synthesised with such 5′ adenylation or this adenylation may be the result of the action of enzymes used in the ligation reaction. For example, T4 RNA ligase uses ATP to adenylate the 5′ end of single-stranded nucleic acid sequences. Whilst this 5′ adenylation is typically precursor to the ligation of the RNA-binding adaptor to the RNA molecule, the presence of the 5′ cap also prevents the actions of the 5′ to 3′ exonuclease, for example, RecJ. Thus, the step of removing any unbound RNA-binding adaptor by contacting the second mixture with a 5′ to 3′ exonuclease may further comprise contacting the second mixture with a 5′ deadenylase. The 5′ deadenylase may be a yeast 5′ deadenylase, for example, the 5′ deadenylase originally isolated from Saccharomyces cerevisiae. The second mixture may be contacted with a 5′ deadenylase prior to being contacted with an exonuclease.

The methods of the invention may further comprise a step of isolating the RNA-binding adaptor-bound cross-linked RBP-RNA. As used herein the term “isolate” refers to a process well known to those of skill in the art in which the RNA-binding adaptor-bound cross-linked RBP-RNA is substantially purified from the other components of the second mixture. Standard methods of isolating the RNA-binding adaptor-bound cross-linked RBP-RNA will be well known to those of skill in the art, for example, gel electrophoresis, chromatography or solid-phase extraction. Typically the RNA-binding adaptor-bound cross-linked RBP-RNA is isolated by gel electrophoresis. Preferably the gel electrophoresis is polyacrylamide gel electrophoresis (PAGE), for example, a tris-borate-EDTA-Urea PAGE, more preferably sodium dodecyl sulfate-PAGE (SDS-PAGE). Through the separation of the components based on size, PAGE is capable of isolating the RNA-binding adaptor-bound cross-linked RBP-RNA based on size. Unbound RNA will not be retained by the gel in view of its small molecular weight. As the skilled person will appreciate, the percentage of polyacrylamide in the gel may be readily selected so as to provide the correct conditions for isolating the RNA-binding adaptor-bound cross-linked RBP-RNA.

The methods of the present invention may further comprise a step of visualising the cross-linked RBP-RNA by detection of the detection means. Visualisation of cross-linked RBP-RNA provides a useful quality control step in the methods of the invention, allowing the presence and integrity of the cross-linked RBP-RNA to be assessed, in particular, against positive and negative control samples. Visualisation also allows the identification of contaminating co-purified complexes. Conventional methods of detecting and, thus, visualising a detection means are well known in the art. The skilled person will be able to select the appropriate detector to detect and thus, visualise, the detection means. By way of non-limiting example, the RBP-RNA may be transferred from an SDS-PAGE gel to a membrane and then visualised. By way of non-limiting example, a fluorescent detection means may be visualised using fluorescence spectrometry whereby the sample is exposed to light at the excitation wavelength of the fluorescent detection means and the fluorescence emitted from the sample is detected.

To further improve the specificity of the methods of the invention, washing steps, typically stringent washing steps, may be included: (i) prior to contacting the purified cross-linked RBP-RNA with the RNA-binding adaptor; (ii) after contacting the purified cross-linked RBP-RNA with the RNA-binding adaptor; (iii) after the addition of the 5′ to 3′ exonuclease (such as Reci); or after any combination of these steps, such as (i) and (ii), (ii) and (iii); or (i), (ii) and (iii).

Typically, the methods of the invention do not comprise a polyadenylation step. In particular, the methods may not comprise a step of polyadenylating the RNA and/or the adaptor.

The present invention provides a method for purifying at least one RNA molecule which interacts with one or more target RNA binding protein, (RBP) comprising the steps of: a. cross-linking the at least one RNA molecule and the one or more RBP in a sample;

b. contacting the sample comprising the cross-linked RBP-RNA with an agent which cleaves RNA to create a first mixture, wherein said agent shortens the RPB-bound RNA;

c. purifying the cross-linked RBP-RNA from the first mixture using an agent that specifically interacts with a component of the cross-linked RBP-RNA;

d. contacting the purified cross-linked RBP-RNA from step c with an RNA-binding adaptor comprising a detection means to create a second mixture, wherein the adaptor binds to the cross-linked RNA;

e. isolating the RNA-binding adaptor-bound cross-linked RBP-RNA;

f. partially digesting the RBP component of the cross-linked RBP-RNA; and

g. purifying the at least one RNA molecule;

wherein optionally said method further comprises the step of preparing the plurality of RNA molecules for high throughput sequencing, wherein steps a to g are typically carried out sequentially.

Said method typically further comprises the following steps:

h. reverse transcription of the at least one RNA molecule to produce a plurality of cDNA molecules;

i. ligation of a cDNA-binding adapter to the 3′ end of the plurality of cDNA molecules; and

j. amplification of the plurality of cDNA molecules.

Again, steps h to j are typically carried out sequentially and subsequent to purification of the at least one RNA molecule (step g as defined above in said passage).

In particular, the present invention provides a method for purifying at least one RNA molecule which interacts with one or more target RNA binding protein, (RBP) comprising the steps of:

a. cross-linking the at least one RNA molecule and the one or more RBP in a sample;

b. contacting the sample comprising the cross-linked RBP-RNA with an agent which cleaves RNA to create a first mixture, wherein said agent shortens the RPB-bound RNA ;

c. purifying the cross-linked RBP-RNA from the first mixture using an agent that specifically interacts with a component of the cross-linked RBP-RNA;

d. contacting the purified cross-linked RBP-RNA from step c with an RNA-binding adaptor comprising a detection means to create a second mixture, wherein the RNA-binding adaptor binds to the cross-linked RNA;

e. removing any unbound RNA-binding adaptor by contacting the second mixture with a 5′ to 3′ exonuclease (e.g. RecJ);

f. isolating the RNA-binding adaptor-bound cross-linked RBP-RNA; and

g. visualising the cross-linked RBP-RNA by detection of the detection means;

thereby purifying at least one RNA molecule which interacts with the one or more target RBP.

Said method steps are typically carried out sequentially from a to g. Optionally said method further comprises the step of preparing the plurality of RNA molecules for high throughput sequencing.

Accordingly, said method may further comprise the following steps:

h. reverse transcription of the at least one RNA molecule to produce a plurality of cDNA molecules;

i. ligation of a cDNA-binding adapter to the 3′ end of the plurality of cDNA molecules; and

j. amplification of the plurality of cDNA molecules.

Again, steps h to j are typically carried out sequentially and subsequent to purification of the at least one RNA molecule (step g as defined above in said passage).

The present invention also provides a method for isolating a plurality of RNA molecules interacting with all RBP contained in a sample, comprising the steps of:

a. cross-linking the plurality of RNA molecules and the RBP in the sample;

b. contacting the sample comprising the cross-linked RBP-RNA with an agent which cleaves RNA to create a first mixture, wherein said agent shortens the RPB-bound RNA ;

c. purifying the cross-linked RBP-RNA from the first mixture using an agent that specifically interacts with protein side chains (e.g. an agent which comprises a carboxyl group);

d. contacting the purified cross-linked RBP-RNA from step c with an RNA-binding adaptor comprising a detection means to create a second mixture, wherein the RNA-binding adaptor binds to the cross-linked plurality of RNA molecules;

e. removing any unbound adaptor by contacting the second mixture with a 5′ to 3′ exonuclease (e.g. RecJ);

f. isolating the RNA-binding adaptor-bound cross-linked RBP-RNA; and

g. purifying the plurality of RNA molecules;

wherein optionally said method further comprises the step of preparing the plurality of RNA molecules for high throughput sequencing.

Said method steps are typically carried out sequentially from a to g, with the step of preparing the plurality of RNA molecules for high throughput sequencing (if included) following step g.

Preparing the plurality of RNA molecules for high throughput sequencing typically comprises the steps: (h) partially digesting the RBP component of the cross-linked RBP-RNA; (i) purifying the at least one RNA molecule; and (j) preparing the at least one RNA molecule for high throughput sequencing.

Accordingly, a method of the invention may further comprise the steps of: (h) partially digesting the RBP component of the cross-linked RBP-RNA; (i) purifying the at least one RNA molecule; and (j) preparing the at least one RNA molecule for high throughput sequencing. Said additional method steps are typically carried out sequentially from h to j, and can follow the step of visualising the cross-linked RBP-RNA.

The skilled person will understand that the expression “partially digesting the RBP component of the cross-linked RBP-RNA” means that the RBP component of the cross-linked RBP-RNA is not completely digested, specifically, that at least one amino acid of the RBP remains cross-linked to the RNA molecule.

The step of partially digesting the RBP component of the cross-linked RBP-RNA may involve the use of a protease. In such instances, the protease hydrolyses peptide bonds of the RBP, thus digesting the RBP. The bond formed between the RBP and RNA during cross-linking is not a peptide bond and, therefore, utilisation of a protease (which cleaves peptide bonds) ensures that at least one amino acid remains cross-linked to the RNA molecule. Partial digestion may therefore be defined as retaining the covalent bond formed by UV crosslinking and at least one amino acid at the direct point of contact between the RBP and the RNA.

Typically, a protease is used to partially digest the RBP component of the cross-linked RBP-RNA. Partial digestion may be defined as removing at least 50%, at least 60%, at least 70%, at least 80%, at least 85%, at least 90%, at least 95%, at least 96%, at least 97%, at least 98%, at least 99% or more of the amino acids of the RBP.

Partial digestion of the RBP may leave a short RBP-derived polypeptide bound at the RBP-RNA interaction site. A short RBP-derived polypeptide may be no more than 30 amino acids, no more than 25 amino acids, no more than 20 amino acids, no more than 15 amino acids, no more than 14 amino acids, no more than 13 amino acids, no more than 12 amino acids, no more than 11 amino acids, no more than 10 amino acids, no more than 9 amino acids, no more than 8 amino acids no more than 7 amino acids, no more than 6 amino acids, no more than 5 amino acids, no more than 4 amino acids, no more than 3 amino acids, or no more than 2 amino acids in length. Preferably said short RBP-derived polypeptide is no more than 15 amino acids, more preferably no more than 10 amino acids, even more preferably no more than 5 amino acids in length. Partial digestion of the RBP may leave a single RBP-derived amino acid bound at the RBP-RNA interaction site. Retaining a short RBP-derived polypeptide or single RBP-derived amino acid allows the binding site to be identified with single nucleotide resolution. In more detail, when reverse transcribing the RNA into cDNA for downstream sequencing, the short polypeptide/single amino acid halts the reverse transcriptase at the site of RBP-RNA interaction. The resulting cDNA is therefore terminated at the binding site.

Means of partially digesting the RBP component of the cross-linked RBP-RNA are well known to those skilled in the art. By way of non-limiting example, the cross-linked RBP-RNA complex may be contacted with a protease to partially digest the RBP component of the cross-linked RBP-RNA complex. Preferably, the protease is proteinase K. The term “proteinase K” as used herein may refer to the proteinase encoded by the PROK gene in Parengyodontium album (Tritirachium album) (UniProt Knowledgebase (UniProtKB) accession number: P06873-1, sequence deposited 1 January 1990). The optimal conditions for proteinase K activity are well established. By way of non-limiting example, partial digestion of the RBP may be carried out by contacting the RBP with proteinase K, optionally in a buffer comprising 10 mM Tris-HCL (pH 7.4), 100 mM NaCl, 1mM EDTA and 0.2% SDS for 60 minutes at 50° C.

The expression “purifying the at least one RNA molecule” is intended to encompass well known processes of substantially separating the at least one RNA molecule from other components in the mixture. The at least one RNA molecule may be purified using phenol extraction. Preferably, the phenol extraction is performed using a phase lock gel column. Alternatively, the at least one RNA molecule may be purified using phenol-chloroform extraction, and/or using a column-based purification method.

Preparing RNA for High-Throughput Sequencing According to Methods of the Invention

As described herein, the invention provides a method for the preparation of at least one RNA molecule for high throughput sequencing. In addition, any of the methods described herein may further comprise additional steps for the preparation of at least one RNA molecule for high throughput sequencing.

Accordingly, the invention provides a method of preparing one or more RNA molecule for high-throughput sequencing comprising: (i) reverse transcription of the one or more RNA molecule to produce a plurality of cDNA molecules; (ii) enzymatic digestion of any unextended reverse transcription primer; (iii) immobilisation of the plurality of cDNA molecules on a solid phase; (iv) ligation of a cDNA-binding adaptor to the immobilised plurality of cDNA molecules; (v) optionally eluting the plurality of cDNA molecules from the solid phase; and (vi) amplification of the plurality of cDNA molecules; wherein optionally the one or more RNA molecule is prepared by a method as described herein.

Preparing the at least one RNA molecule/plurality of RNA molecules for high throughput sequencing typically involves: (i) the reverse transcription of the RNA to produce a plurality of cDNA molecules; (ii) enzymatic digestion of any unextended reverse transcription primer; (iii) immobilisation of the plurality of cDNA molecules on a solid phase; (iv) ligation of a cDNA-binding adaptor to the 3′ end of the immobilised plurality of cDNA molecules; and (v) amplification of the plurality of cDNA molecules. Optionally, an additional step of eluting the plurality of cDNA molecules from the solid phase is included after the ligation of the cDNA-binding adaptor and before the amplification of the plurality of cDNA molecules. An optional step of alkaline hydrolysis to remove the RNA molecules after the reverse transcription step and before the enzymatic digestion step may also be included. Both the elution step and alkaline hydrolysis steps may be included in the methods of the invention.

Methods of preparing the at least one RNA molecule/plurality of RNA molecules for high throughput sequencing may further comprise washing steps, typically stringent washing steps: (i) immediately after immobilisation of the plurality of cDNA molecules on a solid phase; and/or (ii) immediately after ligation of a cDNA-binding adaptor to the 3′ end of the immobilised plurality of cDNA molecules.

The reverse transcription of the RNA to produce a plurality of cDNA molecules may use a reverse transcription primer that is a universal reverse transcription primer. Typically said universal reverse transcription primer is complementary to a common region of the RNA-binding adaptor molecule that is contacted with the purified cross-linked RBP-RNA. In some embodiments, said universal transcription primer comprises a region of between 8 to 18 nucleotides, preferably 15 nucleotides, which are complimentary to a common region of the RNA-binding adaptor molecule that is contacted with the purified cross-linked RBP-RNA. One non-limiting example of such a primer comprises the nucleic acid sequence CGTGTGCTCTTCCGA (SEQ ID NO: 9). Another non-limiting example of such a primer comprise the nucleic acid sequence CGTGTGCTCTTC (SEQ ID NO: 10). Said universal primer may be conjugated to a moiety which aids purification of the resulting cDNA. By way of non-limiting example, the universal primer may comprise a biotin moiety, a streptavidin moiety, an amide moiety, a carboxyl moiety, or a CLICK moiety (for example, the universal primer may have either an alkyne or an azide moiety). Preferably said universal reverse transcription primer is biotinylated, typically at the 5′ end. The moiety may be separated from the nucleic acid sequence of the universal reverse transcription primer by a linker of variable length. Such linkers are well known in the art and include, for example, tetraethyleneglycol (TEG) and polyethyleneglycol (PEG).

Biotinylation of the resulting cDNA molecules allows capture of the cDNA molecules on streptavidin beads, preferably magnetic streptavidin beads.

Methods of the invention may involve a step of alkaline hydrolysis. Alkaline hydrolysis may be used to remove any RNA molecules which remain in the sample. Such RNA molecules can interfere with downstream ligation steps.

Following reverse transcription, the universal primer may be contacted with, and hybridised to its reverse complement. After said hybridisation there exist two populations of reverse transcription-primer: (i) hybrids between unextended primer and its' reverse complement that have blunt ends susceptible to exonuclease digestion; and (ii) hybrids between extended primers and the reverse complement which have a cDNA overhang precluding exonuclease digestion

The enzymatic digestion of any unextended reverse transcription primer is then typically carried out by treating the sample with an exonuclease to digest the double stranded primer DNA. Any appropriate exonuclease may be used, preferably Exonuclease III.

Removal of the unextended reverse transcription primer is advantageous because if the unextended primer remains in the sample, it can subsequently be ligated to the cDNA-binding adaptor to produce an amplifiable artefact. This artefact will then dominate the final library due to its small size and excess.

Immobilisation of the cDNA on a solid phase (e.g. beads) allows a high-stringency wash to be carried out following each of the subsequent steps (or any combination of these steps, or in between every step) in the method of preparing the at least one RNA molecule/plurality of RNA molecules for high throughput sequencing. Such high stringency washes may be carried out using any conventional high stringency conditions standard in the art, preferably in 2M salt (e.g. 2M NaCl). The immobilisation of the cDNA on a solid phase also allows all subsequent steps to be performed on the solid phase to reduce or avoid sample loss. When a biotinylated cDNA-binding adaptor molecule is ligated to the 3′ end of the cDNA, the extended cDNA is preferably immobilised on magnetic streptavidin beads.

As used herein, the term “cDNA-binding adaptor”, refers to an oligonucleotide that is capable of being ligated to the 3′ end of a cDNA molecule. The cDNA-binding adaptor may be composed of DNA nucleotides. Typically, the cDNA-binding adaptor is a single-stranded oligonucleotide. Ligation of a cDNA-binding adaptor to the 3′ end of the cDNA molecule is typically carried out while the cDNA is immobilised (for example, on a solid support). Typically, the cDNA-binding adaptor is between 10 to 40 nucleotides in length. Preferably, the cDNA-binding adaptor is about 27 nucleotides in length. The cDNA-binding adaptor may comprise or consist of a nucleotide sequence selected from: /5Phos/ANNNNNNNAGATCGGAAGAGCGTCGTG/3ddC/(SEQ ID NO: 11); /5Phos/NNNNNNNAGATCGGAAGAGCGTCGTG/3ddC/(SEQ ID NO: 12); and /5Phos/AGATCGGAAGAGCGTCGTG/3ddC/(SEQ ID NO: 13); wherein N may be any nucleotide. cDNA-binding adaptors comprising a stretch of random nucleotides allows PCR duplicates to be determined and counted as a single event rather than many. The skilled person will understand that 5′ phosphorylation of the cDNA-binding adaptor is essential for the ligation reaction. The presence of a dideoxy nucleotide at the 3′ end of the adaptor prevents self-circularisation and catenisation of the cDNA-binding adaptor. Any unligated cDNA-binding adaptor molecules may be removed, e.g. by a high stringency wash (such as in 2M salt).

The plurality of cDNA molecules may then optionally be eluted from the solid phase using any appropriate elution buffer or solution. Preferably, this elution step is carried out in nuclease-free water, metal ion-free water, and more preferably water that is both nuclease-free and free of metal ions. The elution buffer or solution may comprise biotin, preferably excess biotin. The skilled person will understand that the expression “excess biotin” refers to a concentration of biotin that is higher than the concentration of biotin conjugated to the plurality of cDNA molecules. The elution step may be carried out at a high temperature, for example at least 50° C., at least 60° C., at least 70° C., at least 75° C., at least 80° C., at least 85° C. or more, wherein said high temperature is maintained for at least 30 seconds, at least 60 seconds, at least 90 seconds, at least two minutes, at least three minutes, at least four minutes, at least five minutes, at least six minutes, at least seven minutes, at least eight minutes, at least nine minutes, at least ten minutes or more. By way of non-limiting example, a temperature of about 50° C. may be maintained for about six minutes, or a temperature of about 80° C. may be maintained for at least 30 seconds, preferably at least 60 seconds. Any of these temperatures/times may be used in combination with any appropriate elution buffer. Particularly preferred is the use of water that is both nuclease-free and free of metal ions and a temperature of about 80° C. maintained for at least 30 seconds, preferably at least 60 seconds. Preferably the elution step uses a solid phase consisting of streptavidin, preferably using a universal primer with a biotin moiety.

Following the removal of the unligated cDNA-binding adaptors (and elution if an elution step is included), the plurality of cDNA molecules may then be amplified. Typically, this is carried out using indexed forward and reverse PCR primers. Preferably said indexed reverse primers are optionally modified with phosophorothioate bonds at their 3′ end. This phosophorothioate modification is advantageous as it prevents exonuclease digestion of the reverse primer such that a shortened primer is not produced. Shortened primers are disadvantageous as they can lead to artefact production by ligation of any free reverse transcription primer escaping exonuclease III digestion to the cDNA-binding adaptor and amplification thereof. Forward primers for the amplification of the plurality of cDNA molecules are typically between 69 to 90 nucleotides in length, preferably 70 nucleotides in length. Reverse primers for the amplification of the plurality of cDNA molecules are typically between 65 to 90 nucleotides in length, preferably 66 nucleotides in length. Forward primers for the amplification of the plurality of cDNA molecules may comprise of consist of a nucleotide sequence selected from: AATGATACGGCGACCACCGAGATCTACAC[TATAGCCT]ACACTCTTTCCCTACACGACGCTCTTCCGATCT (SEQ ID NO: 14); AATGATACGGCGACCACCGAGATCTACAC[ATAGAGGC]ACACTCTTTCCCTACACGACGCTCTTCCGATCT (SEQ ID NO: 15); AATGATACGGCGACCACCGAGATCTACAC[CCTATCCT]ACACTCTTTCCCTACACGACGCTCTTCCGATCT (SEQ ID NO: 16); AATGATACGGCGACCACCGAGATCTACAC[GGCTCTGA]ACACTCTTTCCCTACACGACGCTCTTCCGATCT (SEQ ID NO: 17); AATGATACGGCGACCACCGAGATCTACAC[AGGCGAAG]ACACTCTTTCCCTACACGACGCTCTTCCGATCT (SEQ ID NO: 18); and AATGATACGGCGACCACCGAGATCTACAC[TAATCTTA]ACACTCTTTCCCTACACGACGCTCTTCCGATCT (SEQ ID NO: 19). Reverse primers for the amplification of the plurality of cDNA molecules may comprise of consist of a nucleotide sequence selected from: CAAGCAGAAGACGGCATACGAGAT[CGAGTAAT]GTGACTGGAGTTCAGACGTGTGCTCTTCCGA*T*C*T (SEQ ID NO: 20); CAAGCAGAAGACGGCATACGAGAT[TCTCCGGA]GTGACTGGAGTTCAGACGTGTGCTCTTCCGA*T*C*T (SEQ ID NO: 21); CAAGCAGAAGACGGCATACGAGAT[AATGAGCG]GTGACTGGAGTTCAGACGTGTGCTCTTCCGA*T*C*T (SEQ ID NO: 22); CAAGCAGAAGACGGCATACGAGAT[GGAATCTC]GTGACTGGAGTTCAGACGTGTGCTCTTCCGA*T*C*T (SEQ ID NO: 23); CAAGCAGAAGACGGCATACGAGAT[TTCTGAAT]GTGACTGGAGTTCAGACGTGTGCTCTTCCGA*T*C*T (SEQ ID NO: 24); and CAAGCAGAAGACGGCATACGAGAT[ACGAATTC]GTGACTGGAGTTCAGACGTGTGCTCTTCCGA*T*C*T (SEQ ID NO: 25). Stars (*) indicate phosphorothioate bonds and the sequences within square brackets ([]) are the index regions.

The amplified plurality of cDNA molecules (also referred to interchangeably herein as the final cDNA library) is typically purified prior to high-throughput sequencing. Purification of the cDNA library may be carried out using any appropriate purification means. Preferably purification is carried out using size-select spin columns. Alternatively, purification may be carried out using gel electrophoresis based size selection, or using size-select solid phase reversible immobilisation (SPRI) beads.

The methods of the invention may comprise a further step of carrying out high-throughput sequencing on the purified cDNA library.

Preferably the methods of the invention use any combination of: (i) the use of a 5′ to 3′ exonuclease (e.g. RecJ) to remove any unligated RNA-binding adaptor molecule; (ii) the use of an exonuclease (preferably exonuclease III) to remove any unextended reverse transcription primer; and (iii) the use of indexed reverse primers modified with 3 phosphorothioate bonds at their 3′ end to amplified the final purified plurality of cDNA molecules (i.e. the cDNA library). Preferably the methods of the invention comprise all these steps. This combination of steps enzymatically eliminates all potential artefacts associated with standard CLIP adaptors which are removed by inefficient and time-consuming gel-based size selection in conventional CLIP protocols.

Preferably, methods of the invention which comprise additional steps for the preparation of at least one RNA molecule for high throughput sequencing involve the immobilisation of the plurality of cDNA molecules on a solid phase. Even more preferably, said immobilisation is achieved by biotinylating the plurality of cDNA molecules (through ligation of a biotinylated cDNA-binding adaptor to the 3′ end of the cDNA as described) and capturing the biotinylated cDNA molecules using magnetic streptavidin beads. Immobilisation of the cDNA on a solid phase also allows for stringent washes to be performed between steps and allows all subsequent steps to be performed on the solid phase, thus reduce or avoid sample loss through transfer of the sample.

More preferably, methods of the invention use at least the combination of: (i) the use of a 5′ to 3′ exonuclease (e.g. RecJ) to remove any unligated RNA-binding adaptor molecule; and (ii) the immobilisation of the plurality of cDNA molecules on a solid phase.

Input Controls for the Methods of the Invention

A method of the invention may further comprise removing a portion of the first mixture immediately after the step of contacting the sample comprising the cross-linked RBP-RNA with an agent which cleaves RNA to create a first mixture, wherein said agent shortens the RBP-bound RNA (i.e. step (b)) and capturing the whole proteome from said portion to provide an input control. In other words, a method of the invention may further comprise removing a portion of the first mixture immediately after the step of contacting the sample with an agent which cleaves RNA to create the first mixture, and capturing the whole proteome from said portion to provide an input control.

The use of an input control is advantageous as it allows for the capture of the whole cell proteome on magnetic beads that are similar to those used for the immuno-precipitation. This includes RBPs in the lysate that are cross-linked to their RNA targets. Accordingly, many RBPs can be entered into the protocol as an input control on magnetic beads, and RBP-RNA complexes overlapping the size range of the RBP-of-interest will be isolated as an important background control for the experiment. The preparation of an input control according to the invention is quick (approximately 5 minutes) and the input control can then be returned to be run alongside experimental samples, whilst all future protocol steps are identical between the experimental samples and input control.

The portion of the first mixture removed may be about 20%, 19%, 18%, 17%, 16%, 15%, 14%, 13%, or 1%. Preferably, the portion of the first mixture removed is between about 6% and about 3%. Even more preferably, the portion of the first mixture removed is about 5%.

The whole proteome may be captured using an agent that specifically interacts with proteins side chains. By way of a non-limiting example, a solid phase with carboxyl groups may be used. By capturing the whole proteome from the portion of the first mixture removed, an input control comprising cross-linked RBP-RNA can be obtained. Typically this input control is processed in parallel to the remainder of the first mixture. In other words, the input control is typically processed using an identical method of the invention as the remainder of the first mixture. Preferably said input control is processed simultaneously to the remainder of the first mixture.

Samples for Methods of the Invention

The methods of the invention may be carried out on any suitable sample comprising at least one RNA molecule and one or more RBP. Said sample may be a tissue sample or sample comprising cells (also referred to herein as a cell sample), preferably a cell sample. When a tissue sample is used, the methods of the invention typically comprise a step of homogenising the tissue, and preferably also lysis of the cells within the tissue sample. When a cell sample is used, the methods of the invention typically comprise a step of lysing the cells to produce a cell lysate. Any appropriate means can be used to homogenise a tissue sample or lyse a cell sample according to the present invention. Standard means and materials for homogenising tissue and lysing cells are known in the art, for example, lysis buffers with or without mechanical disruption with a Dounce homogeniser or automatic homogeniser. The methods of the invention may be carried out using a sample derived from any tissue or cell sample. For example, a cell sample may be obtained from a patient (e.g. a blood sample or tissue biopsy). Alternatively, the cell sample may be obtained from a population of cells grown in vitro, for example in a monolayer culture, suspension culture or three-dimensional culture. Typically, the cell sample is obtained from a monolayer cell culture. One advantage of the present method is that rapid and accurate identification of RBP-RNA interactions can be achieved using small samples as a starting input.

Typically, the methods of the invention may be carried out on a sample comprising at least 100 cells, at least 1000 cells, at least 5,000 cells, at least 10,000 cells, at least 15,000 cells, at least 20,000 cells, at least 30,000 cells, at least 40,000 cells, at least 50,000 cells, at least 100,000 cells, at least 500,000 cells or more. Preferably the methods of the invention may be carried out on a sample comprising at least 10,000 cells, more preferably at least 100,000 cells.

The methods of the invention may be carried out on a sample comprising fewer than 20,000 cells. For example, the sample may comprise 100 to 20,000 cells, 100 to 15,000 cells, 100 to 10,000 cells, 100 to 5,000 cells, 1,000 to 20,000 cells, 1,000 to 15,000 cells, 1,000 to 10,000 cells, 1,000 to 5,000 cells, 5,000 to 20,000 cells, 5,000 to 15,000 cells, or 5,000 to 10,000 cells. The methods of the invention may be carried out on a sample comprising 5,000 to 15,000 cells.

Typically, the methods of the invention may be carried out on a sample comprising between about 50,000 cells to about 5x10⁶ cells. Preferably, method of the invention may be carried out a sample comprising greater than about 1x10⁵ to about 3x10⁶ cells, such as between about 1x10⁶ to about 3x10⁶ cells.

Applications of the Invention

The methods of the invention have utility in multiple applications. For example, the methods of the invention may be used to purify and/or identify one or more RNA molecules which interact with a specific RBP of interest. Said methods may be used to purify and/or identify all the RNA molecules which interact with a specific RBP of interest. Said methods may be used to purify and/or identify a plurality of RNA molecules which interact with all the RBP within a sample.

The methods of the invention may be used to identify micro RNAs (miRNAs) and target molecules that are purified from a given sample by isolating an argonaute protein, or a component of the RNA induced silencing complex (RISC). Once identified, the miRNAs or target molecules can be disrupted/targeted for therapeutic applications or for experimental studies to investigate their function.

The methods of the invention may also be used to identify RNA modifications, for example, 5′ methyl cytosine, by isolating antibodies against said modifications that have been UV crosslinked to RNA targets. RNA modifications can change the way an RNA molecule is processed, how it interacts with RBPs/other RNA molecules, or how it forms secondary structures.

The methods of the invention may be also be used to screen molecules which disrupt the interaction of at least one RNA molecule with one or more RBP. The methods of the invention may be used to screen any molecule which disrupts the interaction of at least one RNA molecule with one or more RBP, for example, a pharmaceutical molecule or non-pharmaceutical (i.e. research) molecule. The sample may be treated with a small molecule pharmaceutical. Alternatively, the sample is treated with a biological pharmaceutical, for example, an antibody or antigen-binding fragment thereof. Alternatively, the sample is treated with an antisense oligonucleotide to block the interaction of at least one RNA molecule with one or more RBP. Interactions between RNA and RBPs may have a causative role in the pathogenesis of a range of diseases (for example, the interaction may lead to cancer cell proliferation). Disruption of such interactions may have the potential to provide therapeutic benefit.

In order to determine whether the molecule being screened disrupts the interaction between an RNA molecule and an RBP, the method for screening molecules may comprise a step of comparing a treated sample with control sample. The control sample may be an untreated control sample, or the control sample may have been treated with an appropriate control substance, for example, the buffer used to solubilise the molecule being screened. Suitable control substances can be determined by the skilled person based on the molecule being investigated in the treated sample.

Accordingly, the present invention provides a method for screening molecules that disrupt the interaction of RNA molecules with RNA-binding proteins comprising the steps of: a) treating the sample with a molecule aimed at disrupting protein-RNA interactions (e.g. oligonucleotide mimic, small molecule compound); b) treating the sample to initiate a covalent bond between all present RNA-binding proteins and their presently interacting RNAs; c) shortening interacting RNAs using an agent that is capable of cleaving RNA bonds; d) purifying the protein-RNA complexes of interest with an agent that specifically interacts with a component of the complex; e) isolating the complex under stringent conditions to remove other non-specific interactions; and f) visualising the protein RNA complexes using fluorescent imaging. The various steps of this screening method may be as described herein. Thus, the invention provides a method for screening molecules which disrupt the interaction of at least one RNA molecule with one or more target RBP, comprising the steps of: (i) treating a sample with a molecule which disrupts protein-RNA interactions; (ii) carrying out a method of isolating/purifying at least one RNA molecules as described herein on the treated sample; and (iii) comparing the treated sample with an untreated control sample. Said method may be used to screen molecules for treating a disease or disorder associated with one or more target RBP.

The methods of the invention also have therapeutic potential in targeting a disease or disorder, which is associated with the function of an RNA-binding protein. The disease or disorder may be any disease or disorder in which the function of an RBP is implicated, for example, cancer, neurological disease, immunological disease, cardiovascular disease, metabolic disease, liver disease or an infection (e.g. a viral infection).

The methods of the invention may also be used to determine gene expression and pre-mRNA processing profile of a sample by assessing the difference between the plurality of isolated RNA molecules in one sample versus the plurality in additional samples. The profile may be used to define a signature of a given sample relative to another. This may represent a signature of a disease, of a treatment, or of a developmental time point.

The methods of the invention may also be used to identify sequences interacting with RBPs of interest. Said sequence may be a motif which interacts with a particular RBP. Typically said motif is at least 1, 2, 3, 4, 5, 6, 7, 8, 9, 10, 11, 12, 13, 14 or 15 nucleotides in length. Typically, said motifs form secondary structures which are recognised by an RBP.

RNA-Binding Adaptors of the Invention

The invention further provides an RNA-binding adaptor comprising a detection means according to the invention.

As described herein, the present inventors have developed RNA-binding adaptors of up to 32 nucleotides in length that surprisingly reduce aberrant binding to non-RNA component of the sample and provide improved visualisation compared to conventional adaptors used in non-isotopic CLIP-based protocols. The synthesis yield of RNA-binding adaptors according to the present invention is also higher and more cost-effective.

Typically the RNA-binding adaptor of the invention is at least 10 nucleotides in length. For example, the adaptor may be 10, 11, 12, 13, 14, 15, 16, 17, 18, 19, 20, 21, 22, 23, 24, 25, 26, 27, 28, 29, 30, 31 or 32 nucleotides in length. Preferably the RNA-binding adaptor is between 15 and 35 nucleotides in length, more preferably between 18 and 35 nucleotides in length, even more preferably between 18 and 32 nucleotides in length.

The provision of adaptors, all with the same 5′ nucleotide, reduces ligation bias in any downstream sequencing steps. Thus, typically the RNA-binding adaptor has an adenine nucleotide at its 5′ position. The adaptor may comprise a nucleotide sequence selected from AGATCGGAAGAGCACACG (SEQ ID NO: 1); A[XXXXXX]NNNAGATCGGAAGAGCACACG (SEQ ID NO: 2); or A[XXXXXXXX]NNNAGATCGGAAGAGCACACG (SEQ ID NO: 3). Alternatively, the RNA-binding adaptor may have any other nucleotide at its 5′ position. Such an adaptor may comprise the following nucleotide sequence: N[XXXXXX]NNNAGATCGGAAGAGCACACG (SEQ ID NO: 4).

As described herein, an RNA-binding adaptor according to the invention may comprise an index section, a barcode section, or both an index section and a barcode section. Preferably the RNA-binding adaptor of the invention comprises an index region and a random barcode region. The index section may be defined as a nucleotide sequence of known base composition, but where this composition varies between different versions of the adapter. In other words, the index section may be defined as a stretch of nucleotides of known sequence. The sequence of the index section may vary between each adapter. The inclusion of an index section of known sequence within an RNA-adapter of the invention allows for sample mixing to occur post-ligation which reduces any technical variability seen. Thus, the index section may comprise from five to ten nucleic acid resides, and typically comprises from five to eight nucleic acid residues, preferably six, seven or eight nucleic acid residues. The barcode section may be defined as a unique molecular identifier composed of a specified length of nucleotides of random sequence composition. Thus, the barcode section may comprise from two to ten random nucleic acid resides, and typically comprises from two to five random nucleic acid residues, preferably three random nucleic acid residues. Thus, an exemplary consensus sequence comprised by an RNA-binding adaptor of the invention is A[XXXXXX]NNNAGATCGGAAGAGCACACG (SEQ ID NO: 2), where X is a nucleic acid residue of the index section and N is a nucleic acid of the barcode section.

The RNA-binding adaptor of the invention may comprise a nucleotide sequence of A[XXXXXX]NNNAGATCGGAAGAGCACACG (SEQ ID NO: 3).

Alternatively, the RNA-binding adaptor may have any other nucleotide at its 5′ position. Such an adaptor may comprise the following nucleotide sequence: N[XXXXXX]NNNAGATCGGAAGAGCACACG (SEQ ID NO: 4).

The RNA-binding adaptor of the invention may be adenylated, typically 5′ adenylated and optionally a deadenylase is used in combination with a 5′ to 3′ exonuclease (such as Reci) to remove any unbound RNA-binding adaptor.

The detection means is typically a fluorophore/fluorescent detection means, preferably a cyanine, more preferably a cyanine with an excitation wavelength of about 675 nm and an emission wavelength of about 694 nm.

The invention also provides the use of an RNA-binding adaptor comprising a detection means according to the invention in a method of CLIP.

Universal Reverse Transcription Primers of the Invention

The invention also provides a universal reverse transcription primer suitable for use in a method of the invention. Said universal reverse transcription primer is typically complementary to the common region of the barcode sequence comprised in the RNA-binding adaptor molecule of the invention that is contacted with the purified cross-linked RBP-RNA. One non-limiting example of such a primer comprises the nucleic acid sequence CGTGTGCTCTTCCGA (SEQ ID NO: 9). Another non-limiting example of such a primer comprise the nucleic acid sequence CGTGTGCTCTTC (SEQ ID NO: 10). Preferably said universal reverse transcription primer is biotinylated, typically at the 5′ end. The biotin moiety may be separated from the nucleic acid sequence of the universal reverse transcription primer by a linker of variable length. A non-limiting example of such a linker is tetraethyleneglycol (TEG).

The invention also provides the use of a universal biotinylated reverse transcription primer according to the invention in a method of CLIP.

Kits of the Invention

The invention further provides a kit comprising: (i) an RNA-binding adaptor of the invention; and/or (ii) a universal reverse transcription primer of the invention, preferably a biotinylated universal reverse transcription primer of the invention; and instructions for using said RNA-binding adaptor and/or primer in a method of cross-linking immunoprecipitation (CLIP). Preferably said kit comprises both an RNA-binding adaptor of the invention; and a universal reverse transcription primer (preferably biotinylated) of the invention.

Definitions

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

The term “variant”, when used in relation to a protein, means a peptide or peptide fragment of the protein that contains one or more analogues of an amino acid (e.g. an unnatural amino acid), or a substituted linkage.

The term “derivative”, when used in relation to a protein, means a protein that comprises the protein in question, and a further peptide sequence. The further peptide sequence should preferably not interfere with the basic folding and thus conformational structure of the original protein. Two or more peptides (or fragments, or variants) may be joined together to form a derivative. Alternatively, a peptide (or fragment, or variant) may be joined to an unrelated molecule (e.g. a second, unrelated peptide). Derivatives may be chemically synthesized, but will be typically prepared by recombinant nucleic acid methods. Additional components such as lipid, and/or polysaccharide, and/or polypeptide components may be included.

Reference to RNA-binding adaptors and/or cDNA-binding adaptors in the present specification embraces fragments and variants thereof, which retain the ability to bind to the target RNA/cDNA in question. Reference to RBPs in the present specification embraces fragments and variants thereof, which retain the ability to bind to target RNA. By way of example, a variant may have at least 80%, preferably at least 90%, more preferably at least 95%, and most preferably at least 97 or at least 99% amino acid sequence homology with the reference sequence (e.g. an RNA-binding adaptors and/or cDNA-binding adaptor of the invention, particularly any SEQ ID NO presented in the present specification which defines a RNA-binding adaptors and/or cDNA-binding adaptor). Thus, a variant may include one or more analogues of a nucleic acid (e.g. an unnatural nucleic acid), or a substituted linkage. Also, by way of example, the term fragment, when used in relation to an RNA-binding adaptors and/or cDNA-binding adaptor, means a nucleic acid having at least ten, preferably at least fifteen, more preferably at least twenty nucleic acid residues of the reference RNA-binding adaptors and/or cDNA-binding adaptor. The term fragment also relates to the above-mentioned variants. Thus, by way of example, a fragment of an RNA-binding adaptors and/or cDNA-binding adaptor of the present invention may comprise a nucleic acid sequence having at least 10, 20 or 30 nucleic acids, wherein the nucleic acid sequence has at least 80% sequence homology over a corresponding nucleic acid sequence (of contiguous) nucleic acids of the reference RNA-binding adaptors and/or cDNA-binding adaptor sequence. These definitions of fragments and variants also apply to other nucleic acids of the invention. In the context of peptide sequences, the term fragment means a peptide having at least ten, preferably at least fifteen, more preferably at least twenty amino acid residues of the reference protein. The term fragment also relates to the above-mentioned variants. Thus, by way of example, a fragment may comprise an amino acid sequence having at least 10, 20 or 30 amino acids, wherein the amino acid sequence has at least 80% sequence homology over a corresponding amino acid sequence (of contiguous) amino acids of the reference sequence.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99% , or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. The terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Preferably the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of pain. A subject can be male or female, adult or juvenile.

A subject can be one who has been previously diagnosed with or identified as suffering from or having a condition in need of treatment or one or more complications related to such a condition, and optionally, have already undergone treatment for a condition as defined herein or the one or more complications related to said condition. Alternatively, a subject can also be one who has not been previously diagnosed as having a condition as defined herein or one or more complications related to said condition. For example, a subject can be one who exhibits one or more risk factors for a condition or one or more complications related to said condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A polypeptide, e.g., a fusion polypeptide or portion thereof (e.g. a domain), can be a variant of a sequence described herein. Preferably, the variant is a conservative substitution variant. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype reference protein. As to amino acid sequences, one of skill will recognize that individual substitutions, deletions or additions to a nucleic acid, peptide, polypeptide, or protein sequence which alters a single amino acid or a small percentage, (i.e. 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of wild-type, e.g. 110%, 125%, 150%, 175%, 200%, 500%, 1000% or more.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure. Typically conservative substitutions for one another include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

A polypeptide as described herein may comprise at least one peptide bond replacement. A single peptide bond or multiple peptide bonds, e.g. 2 bonds, 3 bonds, 4 bonds, 5 bonds, or 6 or more bonds, or all the peptide bonds can be replaced. An isolated peptide as described herein can comprise one type of peptide bond replacement or multiple types of peptide bond replacements, e.g. 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

A polypeptide as described herein may comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser (S), Thr (T), Cys (C), Tyr (Y), Asn (N), Gln (Q), Asp (D), Glu (E), Lys (K), Arg (R), and His (H). A polypeptide as described herein may comprise alternative amino acids. Non-limiting examples of alternative amino acids include D amino acids, beta-amino acids, homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, paraaminophenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, I-amino-1-cyclopentanecarboxylic acid, I-amino-1-cyclohexanecarboxylic acid, amino-benzoic acid, amino-naphthoic acid, gamma-aminobutyric acid, difluorophenylalanine, nipecotic acid, alphaamino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

A polypeptide may be modified, e.g. by addition of a moiety to one or more of the amino acids comprising the peptide. A polypeptide as described herein may comprise one or more moiety molecules, e.g. 1 or more moiety molecules per peptide, 2 or more moiety molecules per peptide, 5 or more moiety molecules per peptide, 10 or more moiety molecules per peptide or more moiety molecules per peptide. A polypeptide as described herein may comprise one or more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization.

Alterations of the original amino acid sequence can be accomplished by any of a number of techniques known to one of skill in the art. Amino acid substitutions can be introduced, for example, at particular locations by synthesizing oligonucleotides containing a codon change in the nucleotide sequence encoding the amino acid to be changed, flanked by restriction sites permitting ligation to fragments of the original sequence. Following ligation, the resulting reconstructed sequence encodes an analogue having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. A polypeptide as described herein may be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA In another aspect, the nucleic acid can be RNA Suitable nucleic acid molecules are DNA, including genomic DNA or cDNA. Other suitable nucleic acid molecules are RNA, including mRNA.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention.

Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M -A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

Alignment score for determining sequence identity

BLOSUM62 table

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

The percent identity is then calculated as:

$$\frac{Total number of identical matches}{\begin{matrix}[ length of the longer sequence plus the number \\ of gaps introduced into the longer sequence \\ in order to align the two sequence ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

Conservative Amino Acid Substitutions

Basic: arginine
lysine
histidine

Acidic: glutamic acid
aspartic acid

Polar: glutamine
asparagine

Hydrophobic: leucine
isoleucine
valine

Aromatic: phenylalanine
tryptophan
tyrosine

Small: glycine
alanine
serine
threonine
methionine

In addition to the 20 standard amino acids, non-standard amino acids (such as 4-hydroxyproline, 6-N-methyl lysine, 2-aminoisobutyric acid, isovaline and a -methyl serine) may be substituted for amino acid residues of the polypeptides of the present invention. A limited number of non-conservative amino acids, amino acids that are not encoded by the genetic code, and unnatural amino acids may be substituted for clostridial polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allothreonine, methyl-threonine, hydroxy-ethylcysteine, hydroxyethylhomo-cysteine, nitroglutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90: 10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labelling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241 :53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30: 10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### SEQUENCE INFORMATION

Exemplary RNA-Binding Adaptors

(SEQ ID NO: 1)
AGATCGGAAGAGCACACG
(SEQ ID NO: 2)
A[XXXXXX]NNNAGATCGGAAGAGCACACG
(SEQ ID NO: 3)
A[XXXXXXXX]NNNAGATCGGAAGAGCACACG
(SEQ ID NO: 4)
N[XXXXXX]NNNAGATCGGAAGAGCACACG
(SEQ ID NO: 5)
A[XXXXXX]NNNAGATCGGAAGAGCACACG/3Cy55Sp/
(SEQ ID NO: 6)
AGATCGGAAGAGCACACG/3Cy55Sp/;
(SEQ ID NO: 7)
A[XXXXXXXX]NNNAGATCGGAAGAGCACACG/3Cy55Sp/
(SEQ ID NO: 8)
N[XXXXXX]NNNAGATCGGAAGAGCACACG/3Cy55Sp/

N may be any nucleotide.

Exemplary Reverse Transcription Primers

(SEQ ID NO: 9)
CGTGTGCTCTTCCGA
(SEQ ID NO: 10)
CGTGTGCTCTTC

Exemplary cDNA-binding adaptors

(SEQ ID NO: 11)
/5Phos/ANNNNNNNAGATCGGAAGAGCGTCGTG/3ddC/
(SEQ ID NO: 12)
/5Phos/NNNNNNNAGATCGGAAGAGCGTCGTG/3ddC/
(SEQ ID NO: 13)
/5Phos/AGATCGGAAGAGCGTCGTG/3ddC/

N may be any nucleotide.

Exemplary Forward Primers for the Amplification of the Plurality of cDNA Molecules

(SEQ ID NO: 14)
AATGATACGGCGACCACCGAGATCTACAC[TATAGCCT]
ACACTCTTTCCCTACACGACGCTCTTCCGATCT
(SEQ ID NO: 15)
AATGATACGGCGACCACCGAGATCTACAC[ATAGAGGC]
ACACTCTTTCCCTACACGACGCTCTTCCGATCT
(SEQ ID NO: 16)
AATGATACGGCGACCACCGAGATCTACAC[CCTATCCT]
ACACTCTTTCCCTACACGACGCTCTTCCGATCT
(SEQ ID NO: 17)
AATGATACGGCGACCACCGAGATCTACAC[GGCTCTGA]
ACACTCTTTCCCTACACGACGCTCTTCCGATCT
(SEQ ID NO: 18)
AATGATACGGCGACCACCGAGATCTACAC[AGGCGAAG]
ACACTCTTTCCCTACACGACGCTCTTCCGATCT
(SEQ ID NO: 19)
AATGATACGGCGACCACCGAGATCTACAC[TAATCTTA]
ACACTCTTTCCCTACACGACGCTCTTCCGATCT

Exemplary Reverse Primers for the Amplification of the Plurality of cDNA Molecules

(SEQ ID NO: 20)
CAAGCAGAAGACGGCATACGAGAT[CGAGTAAT]GTGACTGGAG
TTCAGACGTGTGCTCTTCCGA*T*C*T
(SEQ ID NO: 21)
CAAGCAGAAGACGGCATACGAGAT[TCTCCGGA]GTGACTGGAG
TTCAGACGTGTGCTCTTCCGA*T*C*T
(SEQ ID NO: 22)
CAAGCAGAAGACGGCATACGAGAT[AATGAGCG]GTGACTGGAG
TTCAGACGTGTGCTCTTCCGA*T*C*T
(SEQ ID NO: 23)
CAAGCAGAAGACGGCATACGAGAT[GGAATCTC]GTGACTGGAG
TTCAGACGTGTGCTCTTCCGA*T*C*T
(SEQ ID NO: 24)
CAAGCAGAAGACGGCATACGAGAT[TTCTGAAT]GTGACTGGAG
TTCAGACGTGTGCTCTTCCGA*T*C*T
(SEQ ID NO: 25)
CAAGCAGAAGACGGCATACGAGAT[ACGAATTC]GTGACTGGAG
TTCAGACGTGTGCTCTTCCGA*T*C*T

Stars (*) indicate phosphorothioate bonds and the sequences in the square brackets ([ ]) are the index regions.

The present invention will now be described with reference to the following non-limiting Examples.

### EXAMPLES

### Example 1

### Refinement of Adaptor Design and Ligation Conditions Restores Integrity to Non-Isotopic CLIP

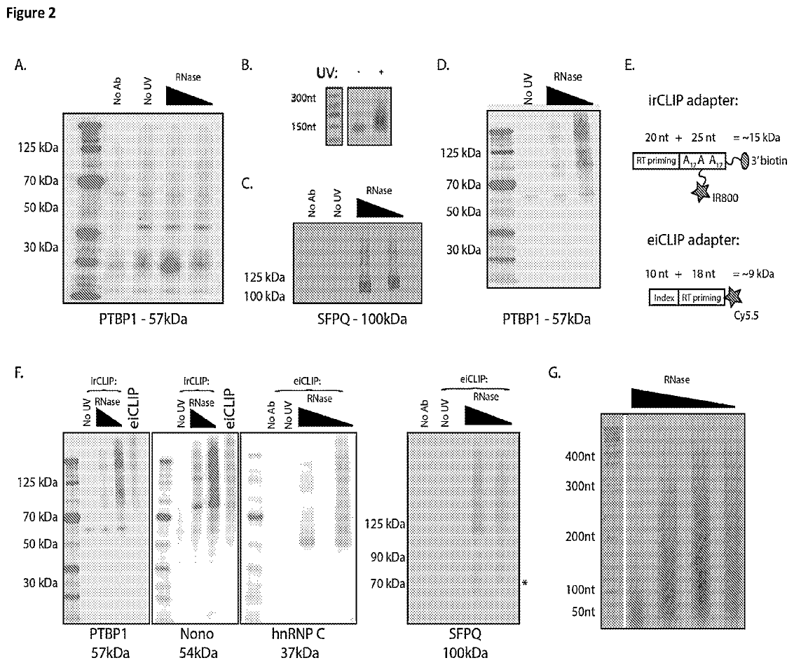

Intense banding of SDS-PAGE analysed RBP-RNA complexes produced was observed using the conventional irCLIP methodology and irCLIP adaptor. This banding was unexpected based on the molecular weight (Mw) of the RBPs being studied (FIG. 2A). These additional bands were present in previously un-assessed negative control samples, consistent between different RBPs, more intense than the expected RNA-derived signal, consistent with the size of enzymes used in previous enzymatic steps, and evident in the positive controls of the original irCLIP study (Zarnegar et al. 2016). Further, cDNA libraries produced with this irCLIP adaptor reveal a dominant adaptor-only by-product that requires removal in irCLIP with post-PCR gel extraction (FIG. 2B). This necessitates a follow-up purification and second library amplification step not incorporated in reported irCLIP timelines.

In view of these results, it was concluded that the conventional 44-nucleotide irCLIP adaptor was non-covalently sticking to all components of the immunoprecipitation reaction, and that this was compounded by limited washing in the irCLIP protocol between enzymatic steps. Supporting this hypothesis, increasing contrast of SDS-PAGE images revealed double bands around the expected molecular weight of certain RBPs under high RNase conditions, and single bands in the no UV that aligned with the lower band of the high RNase sample (FIG. 2C). These are compatible with the adaptor being both ligated to very short RNA in the high RNase condition, and non-covalently sticking to the dominant fraction of non-cross-linked RBP that is immuno-precipitated in both conditions. Increased washing stringency between enzymatic steps and in lysate RNase digestions (rather than on-bead RNase digestions used in irCLIP) also led to a notable improvement in SDS-PAGE quality (FIG. 2D).

To improve integrity and signal intensity of adaptor-ligated RNA, three notable changes were made to develop novel RNA-binding adaptor ligations. First, the RNA-binding adaptor length was reduced to 28 nucleotides by both eliminating redundant nucleotides and by changing the IRDye 800CW DBCO fluorophore, previously used in irCLIP and added via an inefficient CLICK reaction and purification workflow, to a near-infrared Cy5.5 fluorophore incorporated at the 3′ end directly during adaptor synthesis (FIG. 2E). Secondly, we utilised high concentrations of T4 RNA ligase, a non-adenylated adaptor, and the presence of the PEG8000 crowding reagent to enhance ligation efficiency. Thirdly, a post-ligation treatment was introduced using the single-stranded and DNA specific 5′ to 3′ exonuclease, RecJ_{f}, to eliminate retained free RNA-binding adaptor not protected by RNA ligation. When applied together, these modifications changes led to the greatest elimination of unexpected banding patterns in both negative and positive controls samples, and restored smooth signal that appropriately identified different RBPs bound to RNase sensitive RNA (FIG. 2F).

Crucially, in addition to restoring integrity to non-isotopic CLIP analysis, these modifications can be readily incorporated into other CLIP variants that avoid SDS-PAGE quality control (QC) at this crucial step in favour of scalability. Demonstrating necessity for such QC, CLIP of SFPQ with standard conditions led to the co-precipitation of an unidentified RBP component with a lower Mw than expected (FIG. 2F). Notably, this component was not detected by western blotting with the SFPQ antibody. Accordingly, blind cutting at SFPQ's expected Mw would lead to co-purification of longer RNAs derived from this lower component that extend into the SFPQ specific signal, whilst the SDS-PAGE QC of the newly devised methods allows this potential issue to be identified and mitigated. Indeed, conditions can be optimised such that signals do not overlap and specific complexes-of-interest can be isolated. Moreover, the RNase digestion conditions for a given sample can be readily optimised for each experiment by analysing the length distributions of isolated RNAs on high percentage TBE-UREA gels (FIG. 2G). This permits the described method to be conducted without need for non-trivial gel-based size selection used in related methods.

### Example 2

### An Expedited Library Preparation Protocol with Improved Efficiency

Following development of optimal conditions for non-isotopic CLIP analysis (Example 1), the downstream cDNA library preparation workflow was then improved and expedited.

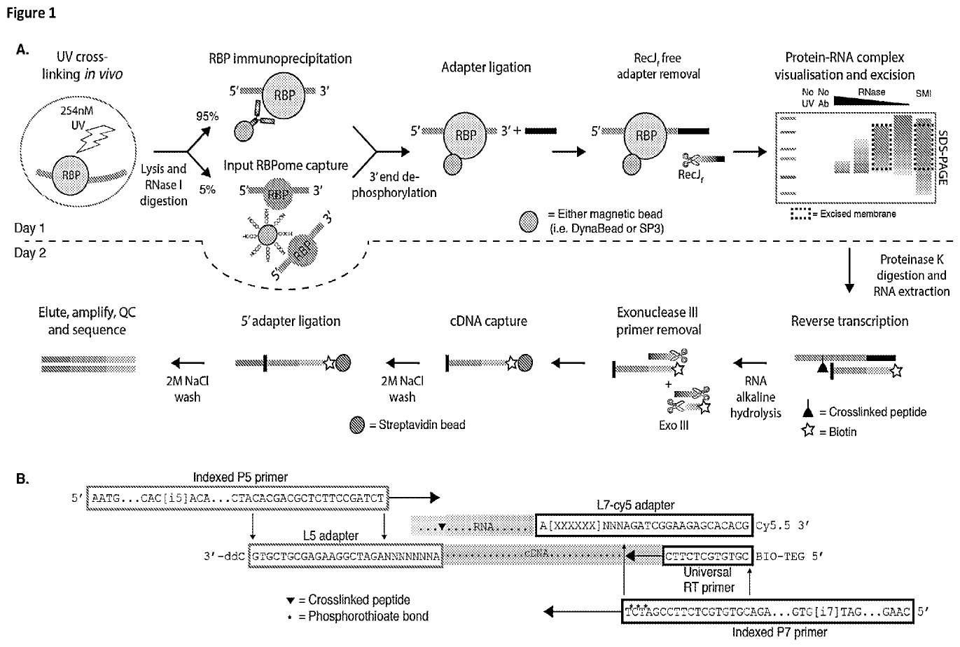

Specifically, we added indexes to the start of the fluorescent RNA-binding adaptor sequences such that sample mixing can occur post-ligation to limit technical variability, eliminated lengthy RNA precipitations with column based purification, used high concentrations of RNA ligase to efficiently ligate a distinct 5′ adaptor containing a unique molecular identifier (UMI) to truncated cDNAs, and ensured final PCR primers were optimised for multiplexing across Illumina sequencing platforms (FIGS. 1A, 1B).

A number of new steps were also introduced to further improve the efficiency. First, as isolated RNAs are barcoded during RNA-binding adaptor ligation, a universal reverse transcription primer with a 5′ biotin moiety was used that allows rapid purification of cDNA on streptavidin coated beads following reverse transcription, subsequent bead-based cDNA-binding ligation, stringent washes after both these steps, and elimination of both precipitations and excessive tube transfers. Once final cDNAs libraries were established, cDNA was then eluted from streptavidin beads via high temperature incubation in cation free water ahead of PCR amplification.

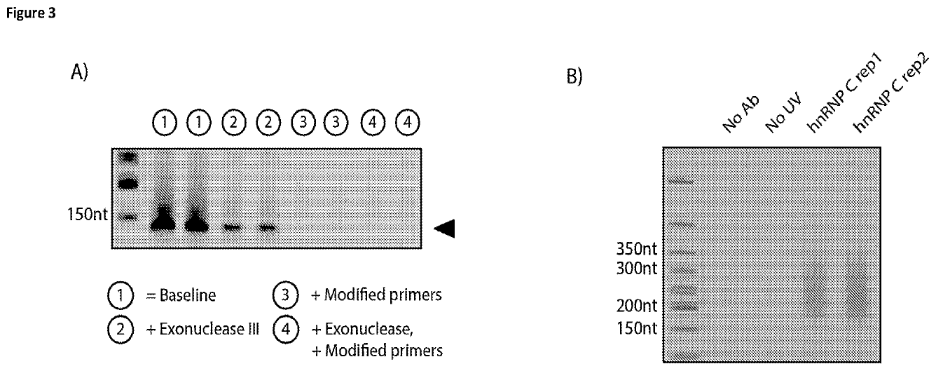

As the new universal reverse transcription primer incorporates a sequence element used in final PCR amplification, a potential amplifiable artefact is derived from direct ligation of any unused reverse transcription primer to the 5′ adaptor carrying the additional final PCR primer site. Similar artefacts are present in existing CLIP protocols, and existing attempts to remove these artefacts relied on time-consuming and error-prone gel purifications of the cDNA (iCLIP) or final PCR amplified (eCLIP, irCLIP) libraries. Further, these previous purification attempts lead to significant loss of material. Accordingly, four preventive steps were implemented in the new method of the invention to eliminate gel purification entirely without material loss (FIGS. 1A, 1B). First, free and un-ligated adapter is removed following the ligation step using the exonuclease, RecJ_{f}, in order to reduce the amount of this artefact template entering the library preparation steps. Adaptor ligated to RNA is protected from this digestion, thus retaining ability to monitor protein-RNA complex formation with high integrity following SDS-PAGE analysis. Second, the reverse complement of the universal primer was annealed following reverse transcription and prior to a new Exonuclease III digestion. Products with a >4 nucleotide extension of cDNA at the 3′ end of the primer are subsequently protected from digestion, whilst both non-extended primers and the reverse complement are degraded. Third, the universal primer requires six nucleotides of extension across the RNA-binding adaptor to create a docking site for primers enabling final PCR amplification. Last, and working in partnership with the third element, indexed PCR primers used for final library amplification incorporated phosphothioate-modified bonds between the last four nucleotides. Accordingly, 3′ to 5′ exonuclease activity of Phusion DNA polymerase was prevented from shortening PCR primers to lengths that are capable of amplifying any remaining universal reverse transcription primer directly ligated to the 5′ adaptor. Crucially, the combination of these last three measures completely eliminates the artefact (FIG. 3A), and leads to artefact free cDNA libraries (FIG. 3B), unlike irCLIP (FIG. 2B).

A variety of RNase conditions were tested to demonstrate that digestion patterns determined with RNA gels mirror the corresponding length distributions of amplified cDNA libraries, and libraries can be created with a broad range of insert sizes that mitigate known potential biases in downstream analysis. Subsequently, RNase conditions were optimised for each batch of samples, and all cDNA libraries produced in absence of any extra size selection. When partnered with additional described modifications, overall cDNA library preparation from purified RNA was subsequently reduced to just a half day, whilst the full protocol produces sequencing ready libraries in just two days (FIG. 1A). Moreover, the improved protocol led to final libraries being amplified from standard starting material at 2-3 PCR cycles less than when a conventional CLIP protocol against the same RBPs.

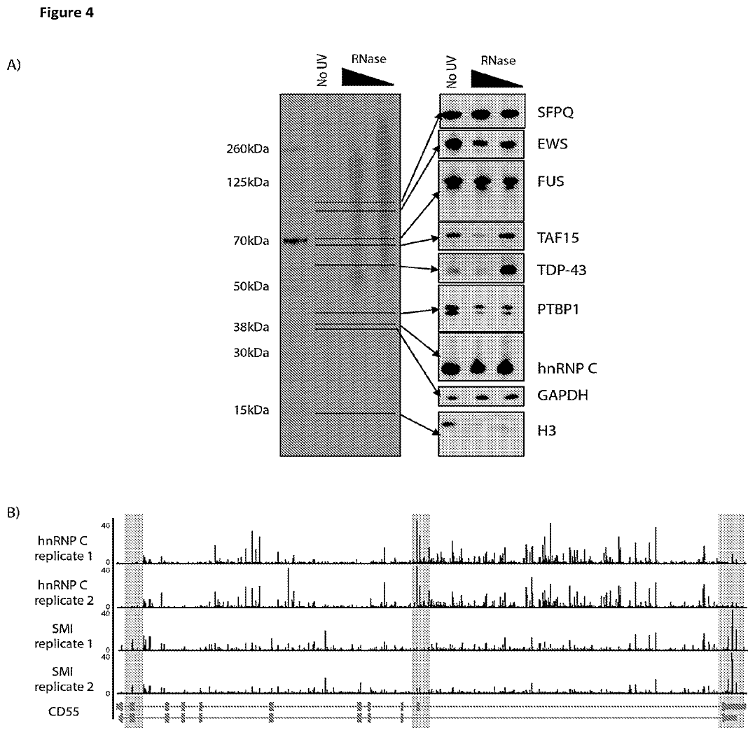

A final improvement was made to the size matched input (SMI) order to control for nonspecific background signal in the same size range of the purified complexes of interest, and to monitor any biases in library preparation. The SMI of the invention captures all RBPs coming from the same size range as the purified complexes of interest then follows identical protocol to experimental samples. This was achieved by exploiting the unbiased capability of SP3 paramagnetic beads to capture proteins for proteomic analysis. It was confirmed that incubating 5% of input lysates with these beads captures both the crosslinked RBPome alongside other cellular proteins (FIG. 4A), and that SMI derived cDNA profiles are distinct from RBPs-of-interest (FIG. 4B). Subsequently, by capturing the RBPome from input samples concomitantly to immunoprecipitations, additional bead-bound samples were seamlessly added to each experiment that then follow identical reactions and library preparation steps (FIG. 1A). Meanwhile, no time is added to the protocol.

### Example 3

### eiCLIP Monitors RBP-RNA Interactions with High Efficiency and Integrity

To validate the enhanced iCLIP (eiCLIP) method of the invention, cDNA libraries were sequenced for hnRNPC made using HeLa cells together with corresponding size-matched inputs. These were subsequently compared to appropriate public datasets from HeLa cells generated using the iCLIP, and irCLIP methods, and to eCLIP datasets of the same protein derived from K562 cells (Table 1). Notably, in order to simplify and standardise downstream eiCLIP computational analysis in future, a pipeline was devised which utilises the publicly available iMaps software for mapping RBP-RNA interactions. Due to compatibility, all datasets from related methods were also processed through this same workflow to facilitate comparisons.

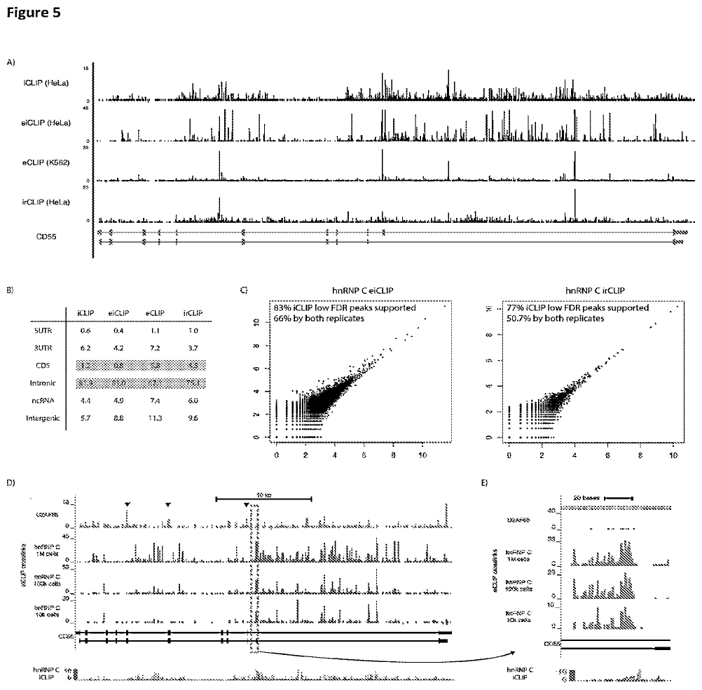

At the individual gene level, it was established that eiCLIP cDNA libraries had good alignment to previously published, well characterised and extensively validated iCLIP libraries (Konig et al. 2011, Nat Struct Mol Biol, 17(7): 909-15, 50:2638; Zarnack et al. 2013, Cell, 152(3): 453-66) (FIG. 5A). In contrast, irCLIP and eCLIP libraries showed distinct cross-linking across transcripts that did not fully overlap eiCLIP or iCLIP to suggest significant technical variation of these approaches (FIG. 5A). Summarising crosslinking positioning across transcripts subsequently revealed that iCLIP and eiCLIP datasets were well matched with ^{˜}1% of crosslinking in mRNA coding sequences, and ^{—}81% of crosslinking in intronic regions. In contrast, eCLIP and irCLIP datasets had notably higher percentages of crosslinking in coding sequences, and notably lower crosslinking in intronic regions (FIG. 56). This again suggests differing profiles of crosslinking between irCLIP and eCLIP to the well validated iCLIP protocol and eiCLIP.

Further, since input cell lines overlapped between iCLIP, eiCLIP and irCLIP datasets, crosslinking events were next evaluated in the different replicates of the eiCLIP and irCLIP approaches to determine whether there was overlap with high confidence clusters established in previous iCLIP studies (Zarnack et al. 2013). This transcriptome comparison notably found that 83% of the high confidence iCLIP clusters were supported by crosslinking events detected in one or more of the two eiCLIP replicates being tested, with 66% then supported by both. This was despite datasets being collected in different labs, by different experimenters and with a time difference of >5 years. In contrast, whilst 77% of the high confidence iCLIP clusters were supported by crosslinking events detected in one or more of the irCLIP replicates being tested, far fewer (50.7%) were now supported by both replicates. Together this implies that eiCLIP and iCLIP identified overlapping binding sites that were highly reproducible, whilst irCLIP had distinct crosslinking profiles that had less agreement across replicates (FIG. 5C). Finally, by performing eiCLIP using different sample inputs, we found that crosslinking sites were reproducible with eiCLIP when using between 10 thousand to 1 million cells. This included both across whole transcripts (FIG. 5D) and at individual validated binding sites (FIG. 5E).

TABLE 1
Comparison of eiCLIP key steps to those of related
CLIP technologies (enhanced CLIP/eCLIP, infrared CLIP/irCLIP,
individual nucleotide resolution CLIP/iCLIP)

| | iCLIP¹ | eCLIP² | irCLIP³ | eiCLIP |
|---|---|---|---|---|
| Labelling | ³²P | — | NIR | NIR |
| Complex QC | Yes | No | Yes | Yes |
| QC integrity | High | — | Low | High |
| Input Ctrl | No | Yes | No | Yes |
| Salt washes | 1M | 1M | 1M | 1M + 2M |
| Duration | 6 d | 4 d | 3 d | 2 d |

1. A method for purifying at least one RNA molecule which interacts with one or more target RNA binding protein, (RBP) comprising the steps of:
a. cross-linking the at least one RNA molecule and the one or more RBP in a sample;
b. contacting the sample comprising the cross-linked RBP-RNA with an agent which cleaves RNA to create a first mixture, wherein said agent shortens the RPB-bound RNA;
c. purifying the cross-linked RBP-RNA from the first mixture using an agent that specifically interacts with a component of the cross-linked RBP-RNA;
d. contacting the purified cross-linked RBP-RNA from step c with an RNA-binding adaptor comprising a detection means to create a second mixture, wherein the adaptor binds to the cross-linked RNA;
e. removing any unbound RNA-binding adaptor by contacting the second mixture with a 5′ to 3′ exonuclease;
f. isolating the adaptor-bound cross-linked RBP-RNA; and
g. visualising the cross-linked RBP-RNA by detection of the detection means;
thereby purifying at least one RNA molecule which interacts with the one or more target RBP.

2. The method of claim 1 further comprising the steps of:
h. partially digesting the RBP component of the cross-linked RBP-RNA, optionally using a proteinase;
i. purifying the at least one RNA molecule; and
j. preparing the at least one RNA molecule for high throughput sequencing.

3. The method of claim 1 or 2, wherein the agent which specifically interacts with a component of the cross-linked RBP-RNA in step c is:
i. an antibody which specifically binds to an RBP of interest;
ii. an antibody which specifically binds to a modification of the RNA of interest; or
iii. a nucleic acid molecule that is homologous to an RNA sequence of interest.

4. The method of any one of the preceding claims, wherein a portion of the first mixture is removed immediately after step b and the whole proteome from said portion captured using an agent that specifically interacts with protein side chains to provide an input control, wherein optionally:
i. the portion of the first mixture removed is about 10%, about 5% or about 1% of the total volume of said first mixture, preferably about 5%; and/or
ii. the input control is processed in parallel to the remainder of the first mixture.

5. A method for isolating a plurality of RNA molecules interacting with all RBP contained in a sample, comprising the steps of:
a. cross-linking the plurality of RNA molecules and the RBP in the sample;
b. contacting the sample comprising the cross-linked RBP-RNA with an agent which cleaves RNA to create a first mixture, wherein said agent shortens the RPB-bound RNA;
c. purifying the cross-linked RBP-RNA from the first mixture using an agent that specifically interacts with protein side chains;
d. contacting the purified cross-linked RBP-RNA from step c with an RNA-binding adaptor comprising a detection means to create a second mixture, wherein the adaptor binds to the cross-linked plurality of RNA molecules;
e. removing any unbound adaptor by contacting the second mixture with a 5′ to 3′ exonuclease;
f. isolating the adaptor-bound cross-linked RBP-RNA; and
g. purifying the plurality of RNA molecules;
wherein optionally said method further comprises: a step of visualising the cross-linked RBP-RNA by detection means between steps (f) and (g) and/or the steps of:
h. partially digesting the RBP component of the cross-linked RBP-RNA, optionally using a proteinase;
i. purifying the at least one RNA molecule; and
j. preparing the at least one RNA molecule for high throughput sequencing.

6. The method of claim 4 or 5, wherein the agent which specifically interacts with protein side chains comprises a carboxyl group.

7. The method of any one of the preceding claims, wherein the sample is a sample comprising cells, wherein optionally the method further comprises a step of lysing the cells to produce a cell lysate, wherein said lysis is performed immediately before step (b).

8. The method of any one of the preceding claims, wherein:
i. the cross-linking is UV cross-linking; and/or
ii the agent which cleaves RNA is a ribonuclease, preferably RNase I.

9. The method of any one of the preceding claims, wherein the agent which specifically interacts with a component of the cross-linked RBP-RNA or the agent that specifically interacts with protein side chains in step c is immobilised on a solid phase, and wherein optionally said solid phase comprises magnetic beads.

10. The method of any one of the preceding claims, which further comprises a washing step under stringent conditions:
i. immediately after step c;
ii. immediately after step d; and/or
iii. immediately after step e.

11. The method of any one of the preceding claims, wherein the RNA-binding adaptor is between 18 and 32 nucleotides in length.

12. The method of any one of the preceding claims, wherein the detection means is a fluorophore/fluorescent detection means, preferably a cyanine, more preferably a cyanine with an excitation wavelength of about 675 nm and an emission wavelength of about 694 nm.

13. The method of any one of the preceding claims, wherein the RNA-binding adaptor comprises a nucleotide sequence selected from:
i.
(SEQ ID NO: 1)
AGATCGGAAGAGCACACG;
ii.
(SEQ ID NO: 2)
A[XXXXXX]NNNAGATCGGAAGAGCACACG;
iii.
(SEQ ID NO: 3)
A[XXXXXXXX]NNNAGATCGGAAGAGCACACG;
iv.
(SEQ ID NO: 4)
N[XXXXXX]NNNAGATCGGAAGAGCACACG;
v.
(SEQ ID NO: 5)
AGATCGGAAGAGCACACG/3Cy55Sp/;
vi.
(SEQ ID NO: 6)
A[XXXXXX]NNNAGATCGGAAGAGCACACG/3Cy55Sp/;
vii.
(SEQ ID NO: 7)
A[XXXXXXXX]NNNAGATCGGAAGAGCACACG/3Cy55Sp/;
viii.
(SEQ ID NO: 8)
N[XXXXXX]NNNAGATCGGAAGAGCACACG/3Cy55Sp/.

14. The method of any one of the preceding claims, wherein the RNA-binding adaptor is 5′ adenylated, and optionally a deadenylase is used in combination with a 5′ to 3′ exonuclease to remove any unbound RNA-binding adaptor.

15. The method of any one of the preceding claims, wherein the 5′ to 3′ exonuclease is RecJ, preferably Recif.

16. The method for purifying at least one RNA molecule which interacts with one or more target RNA binding protein of any one of claims 2 to 4 or 7 to 15, or the method for isolating a plurality of RNA molecules interacting with all RBP contained in a sample of any one of claims 5 to 15, wherein the step of preparing the RNA molecules for high throughput sequencing comprises:
i. reverse transcription of the RNA molecules to produce a plurality of cDNA molecules;
ii. enzymatic digestion of any unextended reverse transcription primer;
iii. immobilisation of the plurality of cDNA molecules on a solid phase;
iv. ligation of a cDNA-binding adaptor to the immobilised plurality of cDNA molecules;
v. optionally eluting the plurality of cDNA molecules from the solid phase; and
vi. amplification of the plurality of cDNA molecules;
wherein optionally the step of preparing the RNA molecules for high throughput sequencing further comprises a step of alkaline hydrolysis to remove the RNA molecules, wherein the step of alkaline hydrolysis is performed between (i) and (ii).

17. A method of preparing one or more RNA molecule for high-throughput sequencing comprising:
i. reverse transcription of the one or more RNA molecule to produce a plurality of cDNA molecules;
ii. enzymatic digestion of any unextended reverse transcription primer;
iii. immobilisation of the plurality of cDNA molecules on a solid phase;
iv. ligation of a cDNA-binding adaptor to the immobilised plurality of cDNA molecules;
v. optionally eluting the plurality of cDNA molecules from the solid phase; and
vi. amplification of the plurality of cDNA molecules;
wherein optionally the one or more RNA molecule is prepared by the method of any one of claims 1 to 16.

18. The method of claim 16 or 17, wherein the reverse transcription uses a revere transcription primer that is a universal biotinylated reverse transcription primer, wherein optionally:
i. said primer comprises a nucleic acid sequence selected from CGTGTGCTCTTCCGA (SEQ ID NO: 9) or CGTGTGCTCTTC (SEQ ID NO: 10);
ii. said primer is biotinylated at the 5′ end; and/or
iii. the oligonucleotide sequence of said primer is separated from the biotin moiety by a linker, preferably tetraethyleneglycol (TEG).

19. The method of any one of claims 16 to 18, wherein:
i. the enzymatic digestion of any unextended reverse transcription primers is carried out using Exonuclease III digestion;
ii. the plurality of cDNA molecules is immobilised using magnetic streptavidin beads;
iii. the plurality of cDNA molecules is eluted from the solid phase in nuclease-free and metal ion-free water at a temperature of at least 50° C.;
iv. the amplification of the plurality of cDNA molecules is carried out by PCR using indexed reverse primers modified with 3 phosphorothioate bonds at the 3′ end;
v. said method further comprises purification of the amplified plurality of cDNA molecules; and/or
vi. said method comprises Exonuclease III digestion of any unextended reverse transcription primers and PCR amplification of the plurality of cDNA molecules using indexed reverse primers modified with 3 phosphorothioate bonds at the 3′ end.

20. The method of claims 2 to 19, which further comprises carrying out high throughput sequencing on the purified cDNA.

21. An RNA-binding adaptor comprising a detection means, as defined in any one of claims 11 to 14.

22. A universal biotinylated reverse transcription primer as defined in claim 18.

23. A kit comprising:
i. an RNA-binding adaptor of claim 21; and/or
ii. a universal biotinylated reverse transcription primer of claim 22;
and instructions for using said RNA-binding adaptor and/or primer in a method of cross-linking immunoprecipitation (CLIP)

24. Use of an RNA-binding adaptor of claim 21 and/or a universal biotinylated reverse transcription primer of claim 22 in a method of cross-linking immunoprecipitation (CLIP).

25. A method for screening molecules which disrupt the interaction of at least one RNA molecule with one or more target RBP, comprising the steps of:
i. treating a sample with a molecule which disrupts protein-RNA interactions;
ii carrying out the method of any one of claims 1 to 20 on the treated sample; and
iii. comparing the treated sample with an untreated control sample;
wherein optionally said method is used to screen molecules for treating a disease or disorder associated with one or more target RBP.
